(19) United States
(12) Patent Application Publication
(10) Pub. No.: US 2025/0114049 A1
(43) Pub. Date: Apr. 10, 2025

(54) PROACTIVE ALERTS AND COORDINATED RESPONSES TO HEALTH EVENTS BY MEDICAL SYSTEMS BASED ON DEVICE USER RESPONSIVITY

(71) Applicant: Medtronic, Inc., Minneapolis, MN (US)

(72) Inventors: Christopher D. Koch, Minneapolis, MN (US); Grant A. Neitzell, Loretto, MN (US); Abhijit P. Jejurkar, Plymouth, MN (US); Paul G. Krause, Mahtomedi, MN (US); Christopher T. House, Pine Island, MN (US)

(21) Appl. No.: 18/727,979

(22) PCT Filed: Jan. 30, 2023
(86) PCT No.: PCT/IB2023/050803

§ 371 Date: Jul. 10, 2024

Related U.S. Application Data
(60) Provisional application No. 63/267,827, filed on Feb. 10, 2022.

(51) Int. Cl. A61B 5/00; G16H 40/20; G16H 80/00

(52) U.S. Cl. CPC A61B 5/747; A61B 5/7282; A61B 5/746; G16H 40/20; G16H 80/00

(57) ABSTRACT

A system comprising processing circuitry configured to receive a wirelessly-transmitted messages from a patient or responder via one of their devices, the messages indicating a verified connection with that patient or responder device and a current location of the patient. After a number of messages, the processing circuitry generates an activity profile for the patient or responder such that in response to a next message, the processing circuitry is configured to determine a level of responsiveness to attribute to the patient or responder and coordinate an emergency response to the patient's current location based on the level of responsiveness.

### FIELD

This disclosure generally relates to systems including medical devices and, more particularly, to monitoring of patient health using such systems.

### BACKGROUND

A variety of devices are configured to monitor physiological signals of a patient. Such devices include implantable or wearable medical devices, as well as a variety of wearable health or fitness tracking devices. The physiological signals sensed by such devices include as examples, electrocardiogram (ECG) signals, electroencephalogram (EEG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect health events based on the physiological signals. Examples of such health events include episodes of cardiac arrhythmia, myocardial infarction, stroke, falls, or seizure. Health events may be short-term or long-term in nature, for example, episodes of physical injuries caused by falling and mental inadequacies caused by dementia, respectively. Example arrhythmia types include cardiac arrest (e.g., asystole), ventricular tachycardia (VT), and ventricular fibrillation (VF). The devices may store ECG and other physiological signal data collected during a time period including an episode as episode data. Such acute health events are associated with significant rates of death, particularly if not treated quickly.

For example, VF and other malignant tachyarrhythmias are the most commonly identified arrhythmia in sudden cardiac arrest (SCA) patients. If this arrhythmia continues for more than a few seconds, it may result in cardiogenic shock and cessation of effective blood circulation. The survival rate from SCA decreases between 7 and 10 percent for every minute that the patient waits for defibrillation. Consequently, sudden cardiac death (SCD) may result in a matter of minutes.

### SUMMARY

In general, the disclosure describes systems, techniques, and devices for patient health monitoring and risk management. The present disclosure describes techniques for sending heartbeat messages (e.g., “heartbeats”) to patients, responders or other interested parties to determine the responsiveness or availability of such individuals to respond to acute health event. Each of a set of heartbeat messages may solicit or request a response message in reply that may be indicative of or usable to determine the responsiveness or availability of such individuals to respond to an acute health event. The heartbeat messages from devices may verify an active connection with a computing service and/or determine one or more attributes regarding patient responsiveness (e.g., connectivity and/or location information) and/or responder responsiveness (e.g., availability and/or location information). The consistency, elapsed time, and other response characteristics associated with the response messages received from the patient or the responder may indicate a degree or level of responsiveness or availability of the patient or the responder. The degree or level of responsiveness or availability of the patient or the responder may be usable to determine who is more or less likely to be responsive to alerts and notifications regarding patient health events and emergency responses.

Consider an example where the patient who has a smart phone regularly communicating “heartbeats” starts moving and leaves their phone behind. Given that the health monitoring service relies on patient data to alert medical professionals, the patient may be putting themself at-risk of a debilitating health event if the health monitoring service receives little to no patient data and/or cannot communicate with the patient. The present disclosure describes a number of other risks associated with leaving the phone behind. In such a situation, the smart phone may generate data memorializing a time period when the patient is not carrying their phone for which the health monitoring service may trigger the transmission of that data via heartbeats. If the patient demonstrates a habit of not having their phone, the health monitoring service may receive a number of heartbeats reflecting that habit and determine that the patient is not carrying their phone around enough for it to be effective as an alerting device.

The entity may mitigate the above behavior via a distribution of alerts to interested parties (including the patient) indicating that the patient is behaving in a risky manner and their health is at a high risk of decline. The entity may further improve upon the above systems, techniques, and devices by configuring the computing service to access various patient data obtained from several data sources (e.g., location-based services and tracking technologies) and coalesce that data into a record of the patient's activities over time (e.g., as defined by the patient's connections and locations). This record, when combined with a model (e.g., a set of rules or criteria), may provide the interested parties with valuable information in evaluating patient data for evidence of responsive behavior(s) or a lack thereof. To further enhance the utility of this information, the computing service may use the record to generate various content for the interested parties to view on their respective devices.

The systems, techniques, and devices of the present disclosure combine respective resource capacities/capabilities of the entity's computing system, the at least one patient device, and the other devices to improve the patient's health, for example, by implementing the proactive safety features and distributing the preventative alerts described herein. To illustrate by way of example, the computing service may be further configured to effectuate coordination of the interested parties for providing medical care to the patient. The systems, techniques, and devices of the present disclosure may direct personnel in administering a response (e.g., a first aid response) to the patient's behavior or acute health event.

In one example, a first computing device includes: processing circuitry; and a memory comprising instructions that, when executed by the processing circuitry, cause the processing circuitry to: at least one of periodically or asynchronously send heartbeat messages to a second computing device of at least one of a patient or a responder, the set of heartbeat messages comprising requests for the patient or the responder to provide user input that causes the second computing device to send response messages that are respectively in response to the set of heartbeat messages; generate, based at least in part on the set of heartbeat messages and the set of response messages, an activity profile of the at least one of the patient or the responder that indicates a level of responsiveness of the patient or the responder to respond to an acute health event of the patient; and perform at least one operation based at least in part on the level of responsiveness of the patient or the responder to respond to the acute health event.

In one example, a computing device includes: an input device; an output device; processing circuitry; and a memory comprising instructions that, when executed by the processing circuitry, cause the processing circuitry to: correlate connectivity and location information received via the input device from computing services running in a plurality of devices across at least one computer network, wherein at least one of the devices comprises a device of a patient that is configured to send an alert of a health event of the patient to at least one other device of the plurality of devices; generate an activity profile of the patient based on the correlated information, wherein the activity profile comprises likely activities of the patient based on time data; based on the activity profile of the patient, determine a level of responsiveness in event of the alert of the health; and communicate, via the output device, information of the level of responsiveness to the patient via the device of the patient.

In one example, a computing device includes: an input device; an output device; processing circuitry; and a memory comprising instructions that, when executed by the processing circuitry, cause the processing circuitry to: correlate availability and location information received via the input device from computing services running in a plurality of devices across at least one computer network, wherein at least one of the devices comprises a device of a patient that is configured to send an alert of a health event of the patient, and wherein at least one of the devices comprises a device of a responder that is configured to receive the alert of the health event of the patient; generate an activity profile of the responder based on the correlated information, wherein the activity profile comprises likely activities of the responder based on time data; based on the activity profile of the responder, determine a level of responsiveness in event of the alert of the health; and communicate, via the output device, information of the level of responsiveness to the responder via the device of the responder.

In one example, a method includes, by processing circuitry: at least one of periodically or asynchronously sending heartbeat messages to a computing device of at least one of a patient or a responder, the set of heartbeat messages comprising requests for the patient or the responder to provide user input that causes the computing device to send response messages that are respectively in response to the set of heartbeat messages; generate, based at least in part on the set of heartbeat messages and the set of response messages, an activity profile of the at least one of the patient or the responder that indicates a level of responsiveness of the patient or the responder to respond to an acute health event of the patient; and perform at least one operation based at least in part on the level of responsiveness of the patient or the responder to respond to the acute health event.

In one example, a method of a computing system coupled to a device of a human user includes, by processing circuitry: send, over a network, a heartbeat to the device of the human user, wherein the heartbeat message corresponds to a connection check with the device of the human user; and receive, over the network, a reply to the heartbeat, wherein the reply comprises an indication of a stable connection between the computing system and the device of the human user.

In one example, a system includes processing circuitry configured to: at least one of periodically or asynchronously send heartbeat messages to a computing device of at least one of a patient or a responder, the set of heartbeat messages comprising requests for the patient or the responder to provide user input that causes the computing device to send response messages that are respectively in response to the set of heartbeat messages; generate, based at least in part on the set of heartbeat messages and the set of response messages, an activity profile of the at least one of the patient or the responder that indicates a level of responsiveness of the patient or the responder to respond to an acute health event of the patient; and perform at least one operation based at least in part on the level of responsiveness of the patient or the responder to respond to the acute health event.

In one example, a non-transitory computer readable storage medium includes program instructions configured to cause processing circuitry to: at least one of periodically or asynchronously send heartbeat messages to a computing device of at least one of a patient or a responder, the set of heartbeat messages comprising requests for the patient or the responder to provide user input that causes the computing device to send response messages that are respectively in response to the set of heartbeat messages; generate, based at least in part on the set of heartbeat messages and the set of response messages, an activity profile of the at least one of the patient or the responder that indicates a level of responsiveness of the patient or the responder to respond to an acute health event of the patient; and perform at least one operation based at least in part on the level of responsiveness of the patient or the responder to respond to the acute health event.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured to detect acute health events of a patient, and to respond to such detections, in accordance with one or more techniques of this disclosure.

FIG. 2 is a block diagram illustrating an example configuration of a patient sensing device that operates in accordance with one or more techniques of the present disclosure.

FIGS. 3A and 3B are each block diagrams illustrating different example configurations of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a flow diagram illustrating an example operation by a health monitoring system that operates in accordance with one or more techniques of the present disclosure.

FIG. 6 is a flow diagram illustrating an example operation by a computing device that operates in accordance with one or more techniques of the present disclosure.

Like reference characters refer to like elements throughout the figures and description.

### DETAILED DESCRIPTION

A variety of types of implantable and external devices are configured detect health events such as arrhythmia episodes based on sensed ECGs and, in some cases, other physiological signals. Some health events may be classified as acute such as sudden cardiac arrest. External devices may run software (e.g., on a remote computing system) to support long-term monitoring by the implantable and external devices configured detect health events. The present disclosure describes a number of technologies to track patient activity (e.g., location) that the external devices can advantageously use to determine a current patient activity and a level of responsiveness (and/or a level of patient risk) associated with that activity. The present disclosure also describes a number of technologies to track responder location and responsiveness, and may coordinate a response to a health event of patient or take other actions based on the responder information.

External devices that may be used to non-invasively sense and monitor ECGs and other physiological signals include wearable devices with electrodes configured to contact the skin of the patient, such as patches, watches, or necklaces. Such external devices may facilitate relatively longer-term monitoring of patient health during normal daily activities. Implantable medical devices (IMDs) also sense and monitor ECGs and other physiological signals, and detect health events such as episodes of arrhythmia, cardiac arrest, myocardial infarction, stroke, and seizure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ or LINQ II™ Insertable Cardiac Monitor (ICM), available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

External devices (e.g., a mobile device such as a smart phone) may be used by the patient (who may or may not have a medical device) as personal devices. One such patient device may be configured to run an application (e.g., a medical application, a browser application, and so forth) operative to exchange data with the computing system of the entity and other devices across multiple networks. Typically, the patient keeps their personal device in their possession while traveling to different locations and at most (if not all) times. This may be because the patient uses the personal device for a wide array of functions, such as those requiring a connection to another computing service. One advantage to having the personal device nearby is that recorded device locations correspond to actual patient locations.

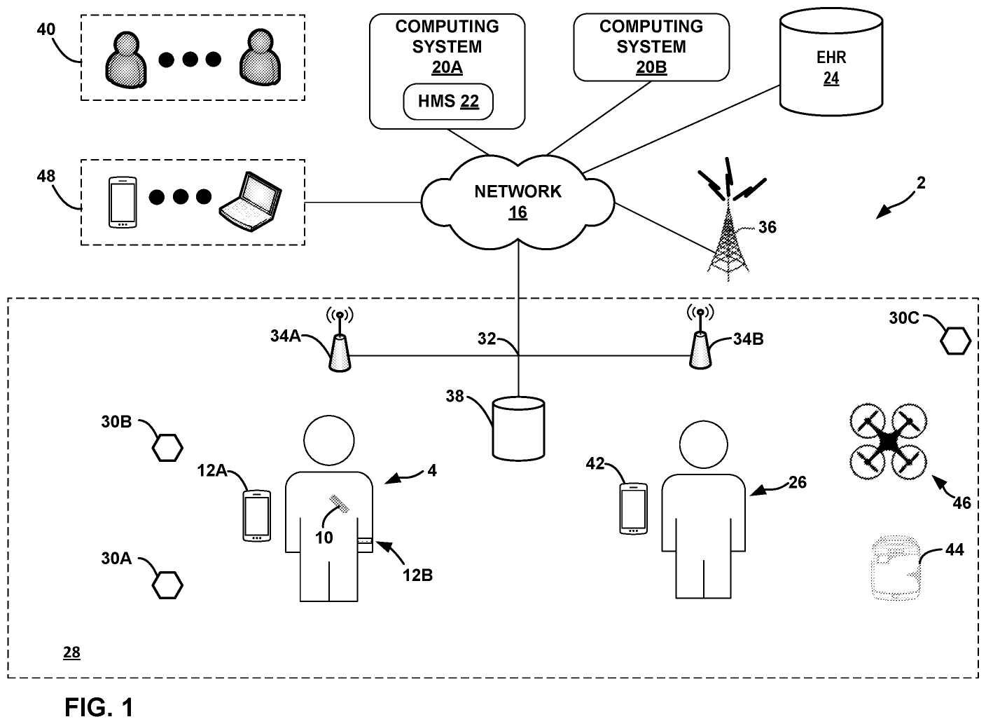

FIG. 1 is a block diagram illustrating an example system 2 configured detect acute health events of a patient 4, and to respond to such detection, in accordance with one or more techniques of this disclosure. As used herein, the terms “detect,” “detection,” and the like may refer to detection of a health event presently (at the time the data is collected) being experienced by patient 4, as well as detection based on the data that the condition of patient 4 is such that they have a suprathreshold likelihood of experiencing the event within a particular timeframe, e.g., prediction of the health event. The example techniques may be used with one or more patient sensing devices, e.g., IMD 10, which may be in wireless communication with one or more patient computing devices, e.g., patient computing devices 12A and 12B (collectively, “patient computing devices 12”). Although not illustrated in FIG. 1, IMD 10 include electrodes and other sensors to sense physiological signals of patient 4, and may collect and store sensed physiological data based on the signals and detect episodes based on the data.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 takes the form of the LINQ™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted patient sensing device, in some examples a system includes one or more patient sensing devices, which may be implanted within patient 4 or external to (e.g., worn by) patient 4.

Patient computing devices 12 (hereinafter computing devices 12) are configured for wireless communication with IMD 10. Computing devices 12 retrieve event data and other sensed physiological data from IMD 10 that was collected and stored by the IMD. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, tablet computer, or smart television. Computing devices 12 may communicate with IMD 10 and each other according to the Bluetooth® or Bluetooth® Low Energy (BLE) protocols, as examples. In some examples, only one of computing devices 12, e.g., computing device 12A, is configured for communication with IMD 10, e.g., due to execution of software (e.g., part of a health monitoring application as described herein) enabling communication and interaction with an IMD.

In some examples, computing device(s) 12, e.g., wearable computing device 12B in the example illustrated by FIG. 1A, may include electrodes and other sensors to sense physiological signals of patient 4, and may collect and store physiological data and detect health events such as cardiac arrhythmia episodes based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A. One or more of computing devices 12 may be configured to communicate with a variety of other devices or systems via network 16. For example, one or more of computing devices 12 may be configured to communicate with one or more computing systems, e.g., computing systems 20A and 20B (collectively, “computing systems 20”) via network 16.

Computing systems 20A and 20B may be respectively managed by manufacturers of IMD 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, computing system 20A implements a computing service (e.g., a location-based service) that receives various datasets indicative of some activity of patient 4 (e.g., connectivity and location information) and/or of one or more of a plurality of candidate responders 40 (e.g., availability and location information). As will be described in greater detail below, HMS 22 monitors activities of patient 4 and proactively alerts one or more candidate responders 40 and/or one or more devices 48.

Computing device(s) 12 may transmit data, including data retrieved from IMD 10, to computing system(s) 20 via network 16. The data may include sensed data, e.g., values of physiological parameters measured by IMD 10 and, in some cases one or more of computing devices 12, data regarding episodes of arrhythmia or other acute health events detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect acute health events for patient 4. In some examples, HMS 22 provides data from EHR 24 to computing device(s) 12 and/or IMD 10 for storage therein and use as part of their algorithms for detecting acute health events.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to detect health events of patient 4 based on data sensed by IMD 10 and, in some cases, other data, such as data sensed by computing devices 12A and/or 12B, and data from EHR 24. In response to detection of an acute health event, IMD 10 may wirelessly transmit a message to one or both of computing devices 12A and 12B. The message may indicate that IMD 10 detected a health event of the patient. The message may indicate a time that IMD 10 detected the health event. The message may include physiological data collected by IMD 10, e.g., data which lead to detection of the acute health event, data prior to detection of the health event, and/or real-time or more recent data collected after detection of the health event. The physiological data may include values of one or more physiological parameters and/or digitized physiological signals. Examples of health events are a cardiac arrest (e.g., a sudden cardiac arrest), a ventricular fibrillation, a ventricular tachycardia, myocardial infarction, a pause in heart rhythm (asystole), or Pulseless Electrical Activity (PEA), acute respiratory distress syndrome (ARDS), a stroke, a seizure, or a fall.

Environment 28 may be a home, office, or place of business, or public venue, as examples. Other devices in the environment 28 of patient 4 may also be configured to output alarms or take other actions to attract the attention of patient 4 and, possibly, a bystander 26, or to otherwise facilitate the delivery of care to patient 4. For example, environment 28 may include one or more Internet of Things (IoT) devices, such as IoT devices 30A-30D (collectively “IoT devices 30”) illustrated in the example of FIG. 1. IoT devices 30 may include, as examples, so called “smart” speakers, cameras, lights, locks, thermostats, appliances, actuators, controllers, or any other smart home (or building) devices. In the example of FIG. 1, IoT device 30C is a smart speaker and/or controller, which may include a display. IoT devices 30 may provide audible and/or visual alarms when configured with output devices to do so. As other examples, IoT devices 30 may cause smart lights throughout environment 28 to flash or blink and unlock doors. In some examples, IoT devices 30 that include cameras or other sensors may activate those sensors to collect data regarding patient 4, e.g., for evaluation of the condition of patient 4.

Computing device(s) 12 may be configured to wirelessly communicate with IoT devices 30 to cause IoT devices 30 to take the actions described herein. In some examples, HMS 22 communicates with IoT devices 30 via network 16 to cause IoT devices 30 to take the actions described herein, e.g., in response to receiving the alert message from computing device(s) 12 as described above. In some examples, IMD 10 is configured to communicate wirelessly with one or more of IoT devices 30, e.g., in response to detection of a health event when communication with computing devices 12 is unavailable. In such examples, IoT device(s) 30 may be configured to provide some or all of the functionality ascribed to computing devices 12 herein.

Environment 28 may be a geographic area in which patient 4 engages in an activity, thereby taking patient 4 to a number of locations within and/or without the geographic area. Environment 28 may have a range to represent an extent HMS 22 (or another computing service) is able to track patient 4, for example, for patient health monitoring and risk management. When patient 4 travels outside of environment 28, HMS 22 may consider patient 4 to be out-of-range and/or at a high level of risk (e.g., due to a lack of responsiveness). HMS 22 may be unable to communicate with patient 4, for example, due to a connection being lost with computing device(s) 12; consequently, if computing device(s) 12 are not communicatively coupled with HMS 22, HMS 22 cannot locate patient 4, for example, to direct responder(s) in case of emergency, and patient 4 may be severely affected by any delay in locating patient 4.

HMS 22 may use a variety of factors to determine if patient 4 is non-responsive to a degree that the patient should be classified as having a lack of responsiveness. HMS 22 may define at least one criterion for determining if patient 4 fails to exhibit even a minimum level of responsiveness. The at least one criterion may include the threshold or another condition to satisfy.

HMS 22 may perform an operation to improve patient 4's responsiveness in view of patient 4's lack of responsiveness. Details for an example operation are included further below the present disclosure. HMS 22 implement a threshold in logic to determine when to perform such an operation. If computing system 20A stores a numerical value to represent patient 4's level of responsiveness for a time period (e.g., an hour), HMS 22 generates the threshold as a single numerical value or a set of values (e.g., forming a pattern) spanning the time period. In the context of periodic heartbeat messages, HMS 22 determines patient 4's level of responsiveness for each hour as a frequency as which computing device 12A returns response messages during that hour. A threshold may be set to a distribution, over 24 hours/day, of hourly frequencies. There may be times during the day (e.g., sleeping hours) when patient 4 is unable to send a response message in reply to a periodic heartbeat and even when able, patient 4 may not respond to every heartbeat and this pattern would be reflected in a patient distribution. HMS 22 of computing system 20A may determine whether the level of responsiveness satisfies the threshold distribution, for example, based on how many patient response frequencies exceed corresponding hourly responses or otherwise, deviate from the pattern. Satisfaction of the threshold may prompt HMS 22 to perform the above operation to improve patient 4's responsiveness.

Environment 28 includes computing facilities, e.g., a local network 32, by which computing devices 12, IoT devices 30, storage system 38, and other devices within environment 28 may communicate via network 16, e.g., with HMS 22, e.g., to upload datasets indicative of connectivity and location information of patient 4. As described herein, HMS 22 may use the uploaded datasets to complete connectivity gaps in other datasets indicative of connectivity and location information of patient 4.

Environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access points 34A and 34B (collectively, “wireless access points 34”) that provide support for wireless communications throughout environment 28. As described herein, wireless access points 34 record datasets indicative of connectivity and location information based on communications of computing device(s) 12. Additionally or alternatively, e.g., when local network is unavailable, computing devices 12, IoT devices 30, and other devices within environment 28 may be configured to communicate with network 16, e.g., with HMS 22, via a cellular base station 36 and a cellular network. Regardless of which technology is employed, wireless access points 34 and/or cellular base station 36 may be configured store datasets indicative of connectivity and location information of patient 4, notwithstanding any previous or existing connection with computing device(s) 12. There are a number of mechanism that can be embodied in local network 32 and/or cellular base station 36 to configure these computing facilities to surreptitiously or indirectly record the above datasets.

In one example, HMS 22 may receive signals (e.g., messages) from computing device(s) 12, computing system 20B, IoT device(s) 30, network access points 34, devices 48, and/or any other device having recorded activity data for patient 4. Some of these signals may be used by HMS 22 as a connection verification for confirming that a particular device of computing device(s) 12 and/or IoT device(s) 30 is functional and operating normally. In some examples, “heartbeat” signals/messages (or, simply, “heartbeats”) request various information of a human (device) user in return to HMS 22 by way of response signals/messages in reply. As explained further below, heartbeat messages may be used to activate response messages in return from computing device 12A of patient 4. Heartbeat messages may be sent periodically or asynchronously to from computing system 20A to computing device 12A of patient 4 or devices of responders 40, and each heartbeat message includes a request for the patient or the responder to provide user input that causes patient 4 or each responder 40 to send response messages that are respectively in response to the heartbeat messages. The user input may include data to indicate a current patient location (e.g., within environment 28) and confirm an active connection (i.e., connectivity) with HMS 22. It should be noted that HMS 22 may receive heartbeats from devices of responders 40. The response messages to heartbeats include signals/messages storing data indicating an availability and a location of each responder 40 for answering an emergency response to health events or non-responsive behaviors of patient 4.

System 2 implements devices, techniques, and/or systems (e.g., via HMS 22) to facilitate prevention and/or detection of health events of patient 4 by coordinating one or more responders 40 and performing other operations to improve an effectiveness of the health event detection/prevention mechanisms, specifically through proactive alerts and notifications to patients/responders whose responsivity negatively impacting the efficacy of system 2. These alerts and notifications inform the patients/responders that their deficient responsivity (e.g., lack of or low responsiveness) is nullifying the health event detection mechanisms. These mechanisms become less effective at alerting patients if the patients cannot perceive the health event alerts. For example, if patient 4 and computing device 12A are in different locations and their distance apart renders ineffective any alarm that computing device 12A can output. As another example, HMS 22 may become ineffective at patient health monitoring if little or no patient data is generated and that may occur if a connection with IMD 10 is lost.

HMS 22 is configured with hardware/software operative to perform accurate patient assessments based on patient input as described herein (e.g., from response messages in reply to “heartbeats”). An example patient assessment for patient 4 may provide various information to attribute to patient 4's activities, such as a level of responsiveness, a level of patient risk, and/or the like. Responsive to determining that patient 4 is at-risk of a health event, HMS 22 directs system 2 to prepare an emergency response to ensure a responder is present at patient 4's location as early as possible. Given that the time succeeding a health event detection is critical to patient mortality, HMS 22's patient assessment for a level of responsiveness is creating extra time for system to complete response preparation without negatively affecting patient 4's health.

HMS 22 may also perform responder assessments based on various responder data to determine a level of responsiveness of each responder 40 in event of a sudden/acute health event of patient 4. HMS 22 enables system 2 to find a qualified responder amongst candidate responders 40. In some examples, HMS 22 may store qualifications data of each candidate responder 40, for example, by tracking that candidate responder 40's progress through certifications, courses, and compliance requirements. HMS 22 may allow system 2 to quickly identify a medical professional best qualified to handle patient 4's high risk level, communicate an instant request to the medical professional to serve as an emergency responder, and then, coordinate a response to patient 4's location. On the other hand, due to an excessive distance between patient 4 and the qualified responder, HMS 22 enables system 2 to coordinate a response from different responder, such as a responder whose location is within a pre-determined proximity of patient 4 and/or a next qualified responder or patient's high risk level. In some examples, HMS 22 may provide system 2 with a system for ranking candidate responders 40 according to different metrics.

By monitoring availability and location information of candidate responders 40, HMS 22 mitigates any potential harm to patient 4 caused by problems in coordinating a response to patient 4's location. For example, there may be instances where none of candidate responders 40 are within a pre-determined proximity of patient 4's location, and in some of those instances, HMS 22 allows system 2 to find an alternate responder amongst devices 48 who is available and within the pre-determined proximity of patient 4's location. In other instances, HMS 22 allows system 2 to conscript bystander 26 into service as an emergency responder for patient 4.

HMS 22 may receive other signals/messages having datasets indicative of connectivity and location information for patient 4. Computing device(s) 12 and/or IoT device(s) 30 may have embedded trackers in hardware and/or software for coalescing different inputs into the connectivity and location information for transmission (e.g., broadcast) ultimately to HMS 22. Some trackers may operate on behalf of a different computing service, such as a location service or a location-based service running on a different computing system than computing system 20A. Via network 16, HMS 22 may access the connectivity and location information for patient 4 from any other computing service.

In general, data store 38 refers to a repository of patient data provided by a plurality sources. The systems, techniques, and devices of the present disclosure advantageously use the patient data to determine a current level of responsiveness of patient 4 and improve upon an effectiveness of its patient health monitoring and health event detection services. One area for improvement may be patient responsivity, particularly, with their device(s) (e.g., alerting devices) as described herein. The present disclosure describes different measures of patient responsiveness to incorporate into the systems, techniques, and devices described herein. As one example measure, the systems, techniques, and devices described herein may evaluate patient engagement with the systems, techniques, and devices providing the patient health monitoring and health event detection services. The systems, techniques, and devices include one or more hardware/software components to compute values for these measures and then, use those values to determine a current level of responsiveness. These systems, techniques, and devices may then identify instances lacking (e.g., an expected degree of) patient device activity. Such instances may specify points-in-time or longer time periods in which patient device activity is lacking. Some instances refer to specific tasks lacking patient device activity. Such tasks may involve returning data in response messages to heartbeats.

Another area for improvement may be an effectiveness of emergency responders in event of acute health events. Criterion for selecting one or more of responders 40 may be based on various types of the patient data, including data indicative of location (i.e., proximity), healthcare records (e.g., previous patient engagement), a current responder activity (e.g., as determined from an activity profile or observed from sensor signals), and other factors or characteristics associated with emergency responses (e.g., standard(s) of patient care). Some systems, techniques, and devices of the present disclosure effectuate reductions in average response time per health event and/or per responder. This may involve selecting responders 40 who are likely to respond quickly to minimize the average response time and/or who are qualified for responding to the health event. Based on various responder data, HMS 22 may identify a responder 40 having a current location within a pre-determined distance of patient 4, a history of short reaction times after notification of a health event, recently responded to a heartbeat from HMS 22, among other criteria.

Selecting responders based on their level of responsiveness may be further based on satisfying one or more criterion. An example criterion may set a threshold whose satisfaction requires a level of responsiveness that exceeds a pre-determined value. This threshold associated with a minimum level of responsiveness expected for an emergency response. Another example criterion may be satisfied if the level of responsiveness is a highest level or responsiveness or in a highest percentile amongst all candidate responders. Yet another example criterion may measure availability of the responder and its effect on the current level of responsiveness. This measure may be based on either a most likely activity or an observed activity of the patient. By comparing this activity with an expected activity of the emergency response, the responder's level of responsiveness may increase or decrease depending on how much time the responder may need for the most likely activity or the observed activity.

In one example, data store 38 refers to a repository of attribute data describing one or more patient activities. The systems, techniques, and devices of the present disclosure advantageously use attribute data for predicting a most likely activity for patient 4 and/or determining a current level of responsiveness of patient 4 based on either the most likely activity or an observed activity. It should be noted that a level of responsiveness may also indicate a level of risk. In one example, HMS 22 may determine the level of risk to patient 4 from the attribute data. Some example attributes include timestamps, patient activity identifiers, location coordinates, and/or the like. Given that patient 4 uses computing device(s) 12 in a personal capacity and/or not just for health monitoring, these devices generate attribute data describing various patent device activities, including application activity, entertainment, communications with a computing service operating on a computing system (e.g., computing system 20B) over network 16, devices 48 with devices of persons engaged in a social relationship with patient 4 (e.g., relatives, friends, and/or the like), and/or so forth.

Any given device capable of communicatively coupling with computing device(s) 12 (which includes device types not illustrated in FIG. 1) may coalesce disparate datasets of patient data into various attributes describing one or more activities of patient 4. Such datasets include information generally indicative of patient behavior in some aspect. Through a variety of mechanisms and methods, devices such as devices 48 may provide data store with sufficient connectivity and location information, for instance, by extracting attributes from metadata, packet header data, and/or the like in corresponding communications between the given device and computing device(s) 12.

Data store 38 may be manufactured in a form configured to maintain a structured arrangement for storing patient data as described herein. FIG. 1 depicts data store 38 as a representation of the connectivity and location information collected, generated, and/or maintained by a plurality of devices in the geographic area of environment 28 or a larger geographic area. HMS 22 may be operative to access various patient data from a plurality of sources, identifies examples of each and every type of connectivity and location information envisioned by the present disclosure, and then, combine the identified examples into the above structured arrangement of data store 38. One example of the structured arrangement may be an activity profile (e.g., a model such as a probability distribution) specific to patient 4 and configured to identify a likely patient activity based on time data.

HMS 22 may configure a number of devices to operate as sources of connectivity and location information. Some devices may operate as part of another computing service (e.g., location service, network service, and/or the like) while other devices may be independent of any computing service. In one example, HMS 22 may configure each of these devices (e.g., computing device(s) 12, IoT device(s) 30, wireless access points 34, devices 48, and devices not illustrated in FIG. 1) with tracking technology (e.g., via a client monitoring application or a background agent) to surreptitiously monitor device activity initiated by patient 4 as well as automated device activity (e.g., background device activity).

Devices such as IoT device(s) 30 and wireless access point(s) 34 may automatically send signals to/receive signals from nearby devices. These signals may be converted into connectivity and location information for patient 4. IoT device(s) 30 and/or wireless access point(s) 34 may operate as part of a service (e.g., a computing service such as a location-based cloud service, a GPS service, a network service, and/or the like) running on computing system 20B. For example, IoT device(s) 30 and wireless access point(s) 34 may broadcast connection requests that are received by computing device(s) 12 and in turn, stored as a dataset of connectivity and location information. Attributes for this dataset may identify a time of receiving/sending the connection request, a location of patient 4 at the time, and whether a connection was made. Computing device(s) 12 may decide to respond with a connection acceptance or not respond at all. Other devices described in the present disclosure may send/receive signals from IoT device(s) 30 and wireless access point(s) 34. Assuming any such device has a wired/wireless connection or a network connection with computing device(s) 12 (e.g., by virtue of being located within a certain distance), both devices may be configured to log connectivity and location information for patient 4 and then, provide such data to HMS 22.

Devices 48 of connections for patient 4 represent a group of connected devices for computing device(s) 12 patient 4. Each of devices 48 currently has a connection (e.g., an ad-hoc connection) or previously had a connection over which data may be exchanged with computing device(s) 12. The present disclosure envisions the data being communicated to be any conceivable dataset of computerized information. Furthermore, a “connection” as described herein refers to any structural configuration of computer or electrical hardware/software operating as a conduit for data such as in the above-mentioned communications. It should be noted that a definite “connection” can be assumed if message data has been successfully transmitted between computing devices (e.g., devices 48 and computing devices 12); although a successful data transmission is not necessary and for most (if not all) devices, a lack thereof is not dispositive of a stable connection existing, confirming existence of a “connection” in any form may require performance of data operations by networking infrastructure (e.g., in computer networking hardware or ad-hoc wireless connectivity software).

Given that patient 4 uses computing device(s) 12 in a personal capacity and/or not just for health monitoring, these devices generate attribute data describing various patent device activities with devices 48 of connections who are persons engaged in a social relationship with patient 4 (e.g., relatives, friends, and/or the like), persons engaged in a health care provider relationship with patient 4 (e.g., caregivers, primary doctor, clinicians, and/or the like), and/or other persons who exchange data with patient 4. As described herein, certain circumstances allow system 2 to consider devices 48 to be potential responders for patient 4; accordingly, data store 38 may store attribute data describing one or more activities between patient 4 and each device 48 and then, upload, to HMS 22, at least a portion of the attribute data in the form of datasets indicative of availability and location information as described herein. Combined with datasets from other sources (e.g., EHR 24), such attribute data may be coalesced by HMS 22 into a record of responder activity for each connection.

HMS 22 may be configured to receive datasets of patient information (e.g., physiological signals from IMD 10 and/or connectivity and location information from a number of sources across network 16) and then, correlate that information to determine a most likely patient activity based on time data. In one example, HMS 22 may coalesce the correlated patient activities into a single profile of patient activity (i.e., a patient activity profile). HMS 22 may implement the patient activity profile to include a probability distribution configured to predict a current patient activity based on a number of features (e.g., a current time). HMS 22 may use the patient activity profile to generate a model (e.g., a machine learning model) configured to determine a risk level of a current patient activity. One example embodiment of the model may be a set of rules for application to various patient data. HMS 22 may apply the set of rules to identify risky patient behavior and upon identifying risky behavior by any patient within environment 28, communicate (at least) one or more messages to notify that patient of the risky behavior and then, proceed to coordinate a proportional response using responders 40 and if unavailable, persons via devices 48 and/or bystander 26.

To prepare for when patient 4 requires immediate medical assistance, HMS 22 may ping responders 40 via their respective devices, for example, by communicating messages to trigger or activate reply messages, and in return, acquire various responder data including availability and location data. The present disclosure describes these messages as initiating “heartbeats” by responders 40, which may refer to a periodic series of signals (e.g., messages) from devices of responders 40 indicating availability as a responder for when patient 4 is in need of immediate medical attention. This message type may also be used to indicate availability of medical professionals for non-urgent care and/or long-term care (e.g., as a caregiver or nurse). Yet another use for this message type may be to indicate availability of any person(s) currently located within a pre-determined proximity of patient 4 (e.g., bystanders).

To perform a connection verification with responders 40, HMS 22 may determine which ones of responders 40 is currently connected to HMS 22 based on who returned a reply message, such as the above-mentioned “heartbeat” message. Receiving the reply message may operate as a confirmation of a stable connection (e.g., a network connection), and over a period of time, HMS 22 may increase a qualitative measure of engagement for any responder(s) 40 who maintained a connection with HMS 22. For any responder(s) 40 who did not maintain a connection (e.g., based on a number of verification failures), HMS 22 may communicate a reminder to each respective responder 40 of their position as an emergency responder.

By providing these “heartbeats” continuously, one or more responder(s) 40 allow HMS 22 to immediately update map content being presented to patient 4 with recorded locations of the qualified responder 40 and therefore, achieve low-latency response times for when patient 4 is at-risk of a health event. Similar to patient 4, devices in environment 28, such as IoT device(s) 30 and wireless access point(s) 34, may activate trackers to monitor movement of the qualified responder 40 and provide HMS 22 with a latest location of the qualified responder 40.

The “heartbeats” described herein may be implemented for patients of HMS 22. HMS 22 may receive heartbeat messages returned from computing device(s) 12 and use each message's content to track patient 4's locations. HMS 22 may combine this information with Internet activity data recorded at network access points 34a to track substantially all of patient 4's data. This activity data may be sufficient for generating an accurate activity profile for identifying a likely patient activity corresponding to time data.

The present disclosure identifies a number of existing technologies that may be used to identify risky behavior by a patient. Systems, techniques, and devices described herein implement tracking technologies to collect a number of attributes generally describing some aspect of the patient's habits. If the patient is likely to visit certain locations over a week, in addition to the patient's home or residence, devices implementing the tracking technologies can collect various data for the patient (e.g., timestamps, device identity, photo/video data, and/or the like). Other devices provide the tracking technologies with information to fill connectivity gaps to the above collected data, such as when the patient leaves their home or residence (which may or may not be geofenced) without their personal device. Cloud computing services may store information generated by applications running on the patient's personal device and those cloud services may be configured to handle requests for any identifiable patient activity data. To illustrate using one of numerous examples, a calendar application may indicate an event with a person having a device 48. The present disclosure describes some (if not all) of these devices as being located within a proximity of the certain patient locations or on a remote computing system and envisions the various patient activity data being collected by these devices as exclusive of no information and inclusive of any information that the systems, techniques, and devices can leverage to enhance the patient's care in at least one aspect.

The following description identifies a number of additional signals providing connectivity and location information for a specific patient. Other signals provide information associated with patient compliance. Patient compliance generally refers to measuring patient reactivity with medical procedures. HMS 22 may receive the above other signals and then, similar to the above datasets of connectivity and location information from a number of devices, generate a model that is configured to determine a risk level of a current patient activity where patient compliance is one example component of that risk level.

One example measure evaluates patient adherence to instructions for using IMD 10 to monitor physiological signals and/or apply a therapy and/or directions for applying a treatment using a device other than IMD 10. Another example measure evaluates patient location over a time period and determines whether patient 4 kept within environment 28 and did not travel out of range of HMS 22. Effective patient monitoring and risk level management may require patient 4 be confined to safe geographic areas and not travel to any high-risk geographic areas. Therefore, HMS 22 may establish the range of environment 28 to confine patient 4, for instance, for their own safety. In some examples, patient 4 may travel beyond the range while continuing to be tracked by HMS 22.

Yet another example measure evaluates patient connectivity with HMS 22 via computing device(s) 12, including any connection gaps or losses. Effective patient monitoring may require patient 4 to be within a limited distance from computing device(s) 12 for a certain amount of time in a time period (e.g., 70% of time between 8 am and 8 pm or as close to 100% as possible). The effectiveness of such patient monitoring can be improved by requiring consistent interaction between patient 4 and HMS 22, for example, via content generated by HMS 22 and presented to patient 4 via computing device 12A. The effectiveness of such patient monitoring can be improved by having patient 4 keep computing device 12A nearby while they sleep. To illustrate, health events such as cardiac arrests often occur at night and/or during patient 4's sleeping hours.

Some signals from computing device(s) 12 carrying information indicative of one or more of the above qualitative measures may be “heartbeats” as described herein (e.g., return messages to pings from HMS 22). Each signal may indicate whether computing device 12A detects patient 4 within the limited distance defined by HMS 22 for a unit of time.

A number of features may be attributed to the surrounding geographic area for patient 4. A qualitative measure corresponding to at least a portion of environment 28 in which patient 4 is currently located may be based on one of these features or a combination of two or more of these features. One example feature may measure bystander reliability within the at least a portion of environment 28. Another example feature may measure caregiver availability for a current time/location, for instance, based (in part) on a caregiver schedule and/or caregiver location within the geographic area of environment 28. Yet another feature may measure emergency medical professional availability for a current time/location, for instance, based (in part) on a proximity of candidate responder(s) 40 to patient 4 from outside of environment 28. Another example feature may account for an effect on a state of the surrounding geographic area of certain conditions, for instance, regarding the caregiver availability, the emergency medical professional availability, and/or the bystander reliability. One of more of these example features (and, possibly, one or more other features) may factor into a determination of the qualitative measure.

HMS 22 may use any number of the above-mentioned qualitative measures for an example (e.g., current) risk level assessment for patient 4. For a current time and location, patient 4 may be attributed a risk level based on a combination of the respective qualitative measures for the surrounding geographic area and patient connectivity/patient compliance. HMS 22 may then compare a threshold risk level to the attributed risk level and based on that comparison, determine whether to communicate proactive notifications to patient 4 and/or any care giver for patient 4.

In one example, HMS 22 may monitor variations in the current risk level for patient 4 over time. In addition to tracking such variations, HMS 22 may determine if any one or more variations is indicative of a high level of patient risk. HMS 22 may further determine a length of time patient 4 maintains the high level of patient risk (e.g., a trend). HMS 22 may then determine an extent (e.g., a magnitude) at which the high level of patient risk exceeds an acceptable (e.g., minimum) risk level for patient 4. If patient 4 is attributed with an abnormally out-of-range and historically high risk level, even when compared to a normalized (e.g., minimum) risk level, HMS 22 may communicate alerts to patient 4 via computing device(s) 12 and/or to bystander 26 via computing device 42. In another example, HMS 22 may activate or trigger alerts to responders 40 and/or devices 48 from patient 4 via computing device(s) 12 and/or via IMD 10. In yet another example, HMS 22 may only activate or trigger an alert to a qualified and available responder amongst responders 40 and/or to a reliable bystander 26 or a reliable connection via device 48. In turn, the qualified/available responder(s) and/or the reliable bystander(s) via their respective devices may reply with query messages regarding patient 4's current health. In another example, a personal caregiver for or a relative of patient 4 could ping computing device(s) 12 to determine whether patient 4 is in an unhealthy state or a health state.

HMS 22 may provide patient 4 via computing devices(s) 12 with information corresponding to any one or more of the above qualitative measures and/or a current level of patient risk based on the one or more of the above qualitative measures. For example, HMS 22 may communicate a notification message informing patient 4 that they met or exceeded the 70% requirement for patient device connectivity or that patient 4 is current located in a high risk geographic area. Patient 4 may be in a low risk geographic area (e.g., within environment 28) but, after patient 4 performs some activity, the same geographic area may transform into a high risk geographic area. Example patient activities including moving away from computing device 12A (e.g., a connective gap), moving out of network 16 (e.g., a connection loss), moving to an area beyond a pre-determined distance from any available responder(s). There are number of existing technologies that HMS 22 may use to determine whether patient 4 is going to be away from their mobile or smartphone, out of range, or in an area without any responders nearby (e.g., within a pre-defined proximity of patient 4). Alternatively, certain conditions may transform into a high risk geographic area without a specific patient activity, such as a lack of qualified responders in environment 28.

Upon HMS 22 identifying a most suitable one of responders 40 to serve as a responder, such as when patient 4 is experiencing a health event or otherwise engaging in highly risky behavior, HMS 22 may generate content for presentation to patient 4 via computing device 12A. HMS 22 may transmit the generated content to computing device 12A coupled to which a display device may be configured to render the content for display. Examples of the content may include map data for the geographic area of environment 28. Other examples of the content may depict environment 28 with a map having an indication of at least one patient location (e.g., latest known location of patient 4). Other examples of the content may include depictions of message content received at HMS 22. The message content generally refers to confirming or rejecting/overriding an emergency state within environment 28, for example, given that a number of responders are within a threshold distance of patient 4.

Outputting such content to patient 4 via computing device(s) 12 may notify patient 4 with text indicating that a qualified responder is on their way to patient 4. The content may include map data to depict recorded locations of the qualified responder. Similar to patient 4, devices in environment 28, such as IoT device(s) 30 and wireless access point(s) 34, may activate trackers to monitor movement of the qualified responder and provide HMS 22 with the latest location of the qualified responder. It should be noted that these trackers are configured to monitor anyone's location(s). If a bystander named Paul is notified of a health event for patient 4, Paul may return a message confirming his movement towards patient 4's location and accepting service as a responder. Patient 4 may use computer device 12A to view a map having indications of Paul's movement towards patient 4's location. The map may further convey location/distance data for anyone else on their way away they are followed by any information about the responder, e.g., if Paul has an AED, an appropriate training level, and is compliant with standard patient care practices.

Based on various information, HMS 22 may generate a ranking system for responders 40. HMS 22 may implement the ranking system to rank responders 40 based on one or more qualitative measures. It should be noted that HMS 22 may implement a number of available technologies for evaluating a medical professional, such as those associated with responder 40, for service as bystander 26 for patient 4. HMS 22 may instrument these technologies for the qualitative measures and determine whether the medical professional is qualified to handle a health event of patient 4.

To prepare for an emergency response, HMS 22 may focus the ranking system on potential responders 40, such as those within a pre-determined proximity of environment 28, available at a present time, and considered reliable and/or qualified. The ranking system may be based on the return of “heartbeats” or the lack thereof. HMS 22 may select one or more of responders 40 if each responder 40 has recently returned a “heartbeat” and has maintained a stable connection with HMS 22 for a period of time. Any responder 40 who remains accessible in throughout an assigned shift and connected via their personal/work device may be considered reliable and therefore, ranked above another responder 40 who is less accessible and/or fails to maintain a consistent connection with HMS 22.

HMS 22 may apply the ranking system to connections of patient 4 via their devices 48 according to one or more metrics. In one example, HMS 22 may compute a rank for each connection and organize devices 48 according to each rank such that any connection of patient 4 who have previously operated as responders (e.g., Patient Associated Responders (PARs)) are ranked higher than other connections. HMS 22 may also apply the ranking system to bystander 26 if no responder 40 is available, reliable, and/or qualified for handling the emergency response.

HMS 22 may also use the ranking system to set a maximum and/or a minimum number of responders to call into service for patient 4 and, possibly, an ordering at which medical professionals/bystanders are put into service for patient 4. HMS 22 may be programmed to handle cases where an insufficient number (e.g., none) of the medical professionals are qualified to be a responder for patient 4, for example, by selecting a top-most ranked medical professional or a highest ranked bystander as bystander 26. HMS 22 may be further programmed to perform an alternative operation to ensure at least one bystander 26 provides patient 4 with timely medical care. Depending on patient 4 and which medical maladies are afflicting that patient, HMS 22 may send different types of medical professionals to provide patient 4 with a complete holistic medical evaluation of their physiological data.

HMS 22 may also use the ranking system to determine which one(s) of responders 40 to call into service for patient 4. Using the ranking system, HMS 22 may assign a qualitative rank (value) to each of responders 40 and if any rank satisfies or exceeds a threshold level of quality associated with successfully managing a risk level of patient 4. HMS 22 may avail a number of qualitative measures to compute a rank for assignment to any given medical professional. One qualitative measure may be based on the medical professional's qualifications with respect to practicing medicine and/or providing medical care. Another qualitative measure may reflect a geographic proximity/distance between the given medical professional and patient 4. HMS 22 may use these same example measures to establish an appropriate quality level threshold. As an alternative, HMS 22 may set the quality level threshold to an arbitrary rank value.

HMS 22 may transmit information to responders 40 for improving upon the timeliness and effectiveness of treatment of the health event of patient 4 by responders 40. HMS 22 may (if needed) transit same or similar information to alternates of responders 40, including people with whom patient 4 has a relationship. In some examples, instead of or in addition to HMS 22 providing an alert message to one or more computing devices associated with responders 40 (e.g., emergency responders such as an Emergency Medical Technician (EMT), a paramedic, or another EMS healthcare provider), computing device(s) 12 and/or IoT devices 30 may be configured to automatically contact EMS, e.g., autodial 911, in response to receiving an alert message from IMD 10. Again, such operations may be cancelled by patient 4, bystander 26, or another user via a user interface of computing device(s) 12 or IoT device(s) 30, or automatically cancelled by computing device(s) 12 based on a confirmatory analysis performed by the computing device(s) overriding the detection of the health event by IMD 10.

HMS 22 may be configured to communicate various information to patient 4, a caregiver of patient 4, any candidate responder(s) 40 within a pre-determined proximity of patient 4, and/or any person serviceable as an emergency responder for patient 4. HMS 22 may communicate a notification indicating patient 4 is (e.g., abnormally) out of range to patient 4 and/or the caregiver of patient 4. HMS 22 may trigger or activate an alert from IMD 10 and/or computing device 12A. The alert may cause output of an alarm to attain the attention of bystander 26.

A notification is a message type that devices generally use to notify someone of patient 4's risky behavior, including patient 4, a caregiver of patient 4, and/or any person serviceable as a responder. An alert is one example notification that devices may use to notify others of a likely occurrence of a health event for patient 4. HMS 22 may receive alerts from computing devices 12. HMS 22 may communicate alerts regarding patient 4 to responders 40 and/or devices 48. Responders 40 and/or devices 48 may return messages indicating availability as a responder for when patient 4 is in need of immediate medical attention. Responders 40 and devices 48 may receive reminders to return the messages indicating their availability; in one example, devices 48 may receive more reminders than responders 40.

HMS 22 may be configured to transmit an alert message to computing device 42 of bystander 26, which may improve the timeliness and effectiveness of treatment of the health event of patient 4. Computing device 42 may be similar to computing devices 12 and computing devices 48, e.g., a smartphone. In some examples, HMS 22 may determine that bystander 26 is proximate to patient 4 based on a location of patient 4, e.g., received from computing device(s) 12, and a location of computing device 42, e.g., reported to HMS 22 by an application implemented on computing device 42. In some examples, HMS 22 may transmit the alert message to any computing devices 42 in an alert area determined based on the location of patient 4, e.g., by transmitting the alert message to all computing devices in communication with base station 36.

In some examples, the alert message to bystander 26 may be configured to assist a layperson in treating patient 4. For example, the alert message to bystander 26 may include a location (and in some cases a description) of patient 4, the general nature of the health event, directions for providing care to patient 4, such as directions for providing cardio-pulmonary resuscitation (CPR), a location of nearby medical equipment for treatment of patient 4, such as an automated external defibrillator (AED) 44 or life vest, and instructions for use of the equipment. In some examples, computing device(s) 12, IoT device(s) 30, and/or computing device 42 may implement an event assistant configured to use natural language processing and context data to provide a conversational interface for bystander 42. The assistant may provide bystander 26 with directions for providing care to patient 4 and respond to queries from bystander 26 about how to provide care to patient 4.

Generally, the alerts or alert messages described herein may include any of the data collected by IMD 10, computing device(s) 12, and IoT device(s) 30, including sensed physiological data, time of the health event, location of patient 4, and results of the analysis by IMD 10, computing device(s) 12, IoT device(s) 30, and/or HMS 22. For example, HMS 22 may be configured to transmit alert messages to one or more devices 48 associated with one or more responders 40 via network 16. Responders 40 may include EMTs, paramedics, and other medical professionals trained for first response and under a healthcare provider such as EMS and hospitals; some responders 40 may be associated with particular departments within a hospital, such as an emergency department, catheterization lab, or a stroke response department. Computing devices 48 generally belong to any person with whom patient 4 has some form of a relationship and/or from whom patient 4 may receive aid. Computing devices 48 may include smartphones, desktop, laptop, or tablet computers, or workstations associated with such systems or entities, employees of such systems or entities, bystanders within a pre-determined proximity of patient 4, or qualified medical professionals and other potential caregivers for patient 4.

Between separate alerts notifying patient 4 of different health events, HMS 22 may contact different ones of responders 40. HMS 22 may select a different responder 40 for each health event based on various factors. As one example factor, the specific qualifications attained by responder 40 may render that medical professional to be (most) suitable for treating/healing patient 4 of a particular health event. In some examples, the particular health event (e.g., a sudden cardiac arrest or a cardiac episode of an arrhythmia) may be detected by native functionality within IMD 10 or another medical device. In other examples, the particular health event (e.g., dementia) may be detected using an application running in computing device(s) 12 and/or based on the same signals from IMD 10 and/or additional signals.

In addition to differentiating the medical care response as well as the actual bystander 26 directed to patient 4 for separate health events, HMS 22 may be further configured to alter the (e.g., emergency) medical care response and/or bystander 26 for a specific heath event. HMS 22 may select bystander 26 and direct that medical professional or bystander to apply certain medical treatments to patient 4 to mitigate or cure a current malady. Contemporaneous and/or subsequent patient 4's activities (e.g., including changes in patient 4's location and/or patient 4's physiology) may cause HMS 22 to select a different person as bystander 26 and/or modify the medical treatments applied to patient 4.

In some examples, HMS 22 may mediate bi-directional audio (and in some cases video) communication between responders 40 and patient 4 or bystander 26. Such communication may allow responders 40 to evaluate the condition of patient 4, e.g., through communication with patient 4 or bystander 26, or through use of a camera or other sensors of the computing device or IoT device, in advance of the time they will begin caring for the patient, which may improve the efficacy of care delivered to the patient. Such communication may also allow the care providers to instruct bystander 42 regarding first responder treatment of patient 4.

In some examples, HMS 22 may control dispatch of a drone 46 to environment 28, or a location near environment 28 or patient 4. Drone 46 may be a robotic device, such as unmanned aerial vehicle (UAV) or another robot. Drone 46 may be equipped with a number of sensors and/or actuators to perform a number of operations. For example, drone 46 may include a camera or other sensors to navigate to its intended location, identify patient 4 and, in some cases, bystander 26, and to evaluate a condition of patient. In some examples, drone 46 may include user interface devices to communicate with patient 4 and/or bystander 26. In some examples, drone 46 may provide directions to bystander 26, to the location of patient 4 and regarding how to provide first responder care, such as CPR, to patient 4. In some examples, drone 46 may carry medical equipment, e.g., AED 44, and/or medication to the location of patient 4.

In some examples, HMS 22 may control dispatch of drone 46 (e.g., as an in-home robot) to a home of patient 4. In general, drone 46 may secure the home as a safe environment for patient 4. If emergency care is needed, drone 46 may be equipped to perform certain medical procedures. For example, drone 46 may be configured to access an airway and start ventilation. As another example, drone 46 may move patient 4 away from harm (e.g., by turning off bath water, removing patient from bath, moving patient away a fire or an electrical hazard, opening/unlocking doors, accessing car garage controls, and/or facilitating police and emergency service access to patient 4). HMS 22 may program the robotic device to delivery therapy or recommend therapy based on a cohort analysis of patient 4's current disease state and sensed physiological data. Drone 46 may also be operative to put the patient into a hypothermic state, if needed. In some instances, drone 46 may confirm whether there are witnesses, and activate normal operation if none are detected. Drone 46 may be configured to summon a bystander to support CPR on patient 4. Drone 46 may be configured to make an ECG or pulse measurement or may operate as an AED by touching two parts of patient 4's body with extendable arms having electrodes.

In examples in which computing device(s) 12 are configured to perform an health event (e.g., confirmation) analysis, computing device(s) 12 may transmit alert messages to HMS 22 and/or IoT devices 30 in response to detecting (e.g., and confirming or rejecting) the health event. In some examples, computing device(s) 12 may be configured to transmit the alert messages prior to completing the confirmation analysis, and transmit cancellation messages in response to the analysis overriding the detection of the health event by IMD 10. HMS 22 may be configured to perform a number of operations in response to receiving an alert message from computing device(s) 12 and/or IoT device(s) 30. HMS 22 may be configured to cancel such operations in response to receiving a cancellation message from computing device(s) 12 and/or IoT device(s) 30.

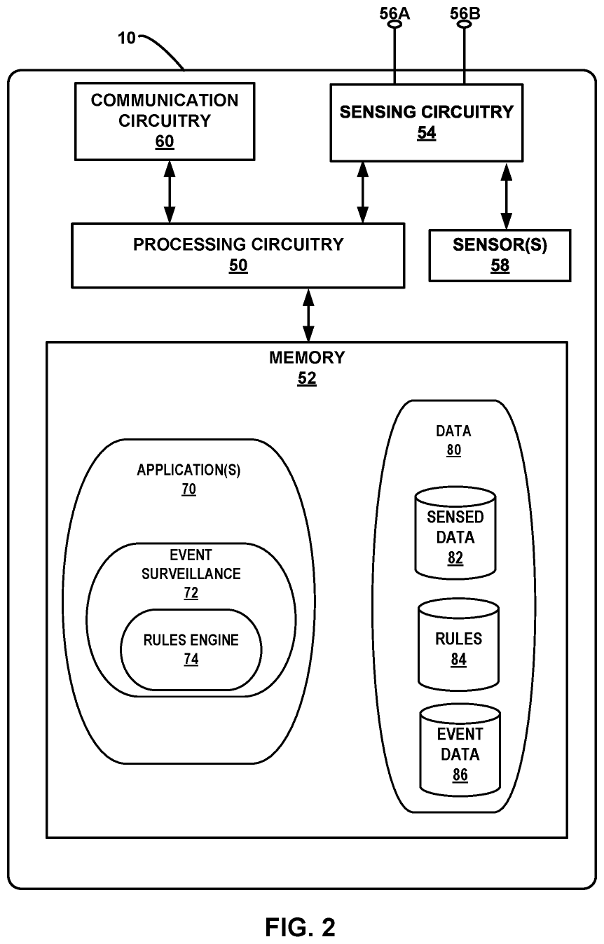

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes one or more sensors 58, such as one or more accelerometers, microphones, optical sensors, temperature sensors, and/or pressure sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications 70 executable by processing circuitry 50, and data 80. Applications 70 may include health event surveillance application 72. Processing circuitry 50 may execute event surveillance application 72 to detect a health event of patient 4 based on combination of one or more of the types of physiological data described herein, which may be stored as sensed data 82. In some examples, sensed data 82 may additionally include data sensed by other devices, e.g., computing device(s) 12, and received via communication circuitry 60. Event surveillance application 72 may be configured with rules engine 74. Rules engine 74 may apply rules 84 to sensed data 82. Rules 84 may include one or more models, algorithms, decision trees, and/or thresholds. In some cases, rules 84 may be developed based on machine learning.

As examples, event surveillance application 72 may detect a cardiac arrest, a ventricular fibrillation, a ventricular tachycardia, a cardiac pause of asystole, pulseless electrical activity (PEA), or a myocardial infarction based on an ECG and/or other physiological data indicating the electrical or mechanical activity of heart 6 of patient 4 (FIG. 1). In some examples, event surveillance application 72 may detect stroke based on such cardiac activity data. In some examples, sensing circuitry 54 may detect brain activity data, e.g., an electroencephalogram (EEG) via electrodes 56, and event surveillance application 72 may detect stroke or a seizure based on the brain activity alone, or in combination with cardiac activity data or other physiological data. In some examples, event surveillance application 72 detects whether the patient has fallen based on data from an accelerometer alone, or in combination with other physiological data. When event surveillance application 72 detects a health event, event surveillance application 72 may store the sensed data 82 that lead to the detection (and in some cases a window of data preceding and/or following the detection) as event data 86.

In some examples, in response to detection of a health event, processing circuitry 50 transmits, via communication circuitry 60, event data 86 for the event to computing device(s) 12 (FIG. 1). This transmission may be included in a message indicating the health event, as described herein. Transmission of the message may occur on an ad hoc basis and as quickly as possible. Communication circuitry 60 may include any suitable hardware, firmware, software, or any combination thereof for wirelessly communicating with another device, such as computing devices 12 and/or IoT devices 30.

In addition to driving patient health monitoring and heath event detection, the present disclosure identifies a number of ways that IMD 10 provides the systems, techniques, and devices described herein with support towards their efforts, for example, in patient risk management support. IMD 10 may generate a variety of patient information (e.g., physiological signals including sensor data 82 and physiological parameters determined by rules engine 74 via an application of rules 84).

HMS 22 of FIG. 1 may be configured to determine the current level of risk for patient 4 based on an activity predicted via an activity profile (e.g., a model). As described herein, examples of HMS 22 may perform a risk level assessment for patient 4 by leveraging the connectivity and location information described in detail for FIG. 1 for predicting a current activity of patient 4. HMS 22 may advantageously use data received/retrieved from IMD 10 to improve upon the above-mentioned patient activity prediction/risk level assessment. In addition to or instead of the activity profile for determining a (most) likely patient activity, HMS 22 may observe, from the received/retrieved data, signal data indicative of current patient behavior and determine which activity is most likely for patient 4. HMS 22 may combine multiple predictions into a single prediction.

HMS 22 may advantageously utilize observed patient behaviors from one or more data sources, such as from connectivity and location information in data store 38 and/or patient information in IMD 10, for accurately estimating a level of risk that logically can be attributed to patient 4 of FIG. 1. Based on determining a reasonable prediction for the most likely patient activity at a current/present point-in-time and possibly, one or more other factors, HMS 22 may determine a current level of patient risk. HMS 22 may improve upon conventional patient risk assessment through a variety of methods/mechanisms. HMS 22 may measure “accuracy” (and therefore, improvements in accuracy) through different means. For example, HMS 22 may implement a model to predict the patient's risk level and then, perform example steps to improve upon the accuracy of that model, for instance, in terms of specificity and/or sensitivity.

For any given patient, HMS 22 may achieve a more accurate determination of a current risk level by performing one or more example steps as described herein. Applying different factors most likely will have at least a modicum of influence on the current level of patient risk. Adding more factors and/or selecting more informative features to use as factors of the current level of patient risk also have an effect on the logic used for the determination of the current patient risk level. Therefore, deciding which data point(s) to use for the patient risk assessment may substantially affect which level of risk is determined. It should be noted that HMS 22 may perform additional example steps towards achieving a more accurate patient risk assessment.

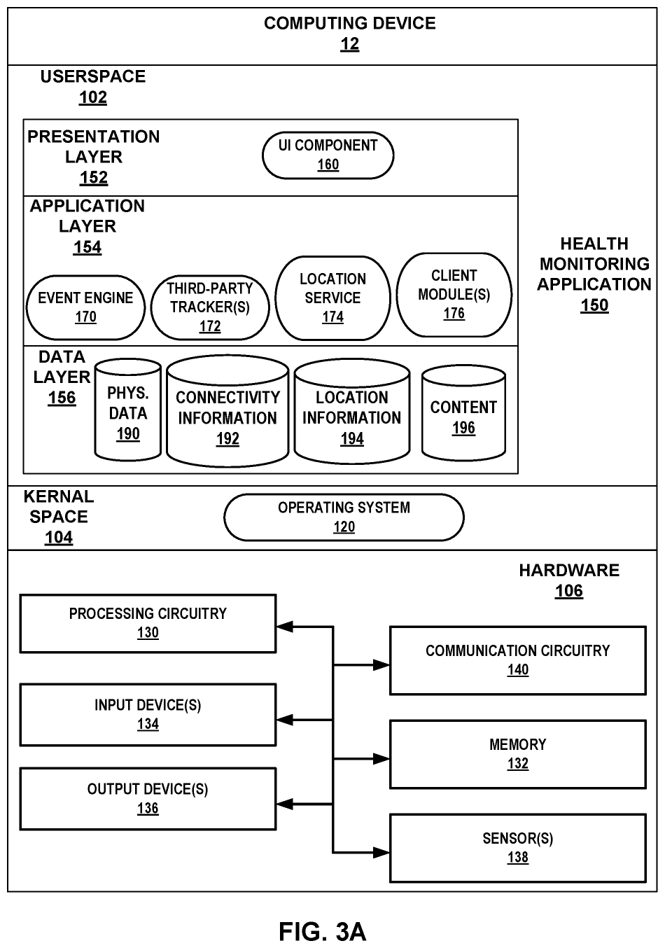

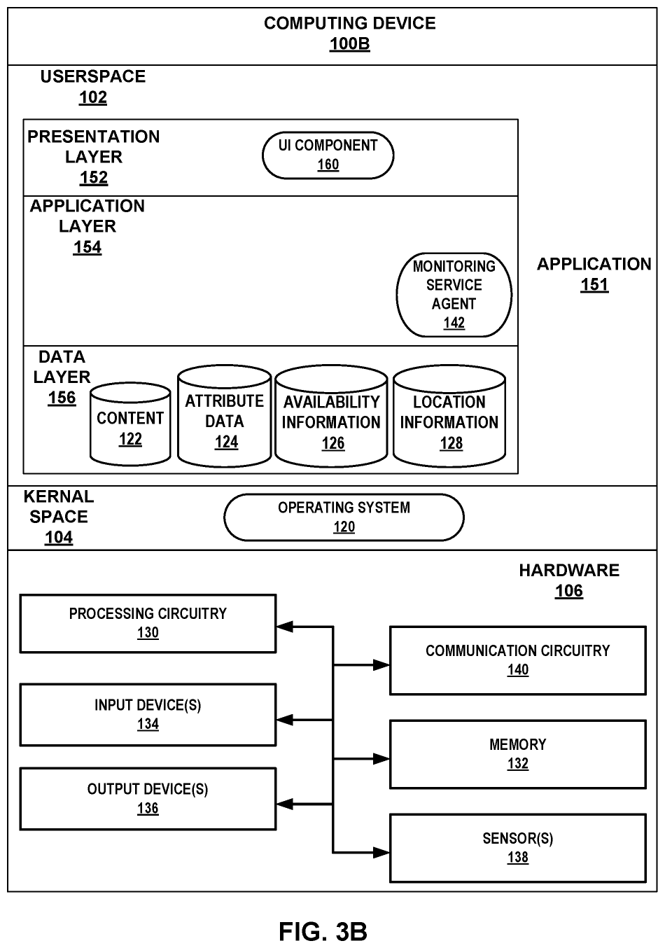

FIG. 3A is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a smart television, a laptop, a tablet computer, a personal digital assistant (PDA), a smartwatch or other wearable computing device.

It should be noted that computing device 12 is capable of providing “heartbeats” as described herein, for example, to provide HMS 22 with first datasets indicative of connectivity and location information of a patient. In some examples, IoT devices 30 may be configured similarly to the configuration of computing device 12 illustrated in FIG. 3A and capable of generating second datasets indicative of connectivity and location information of the patient. Combining both datasets may provide a complete picture of the patient's activity throughout a day.

As shown in the example of FIG. 3A, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3A, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3A.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random-access memories (DRAM), ferroelectric random-access memories (FRAM), static random-access memories (SRAM), and other forms of volatile memories known in the art. Memory 132, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 134 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, 3-axis accelerometers, an optical sensor, an impedance sensor, a temperature sensor, a pressure sensor, a heart sound sensor, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3A, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, an event engine 170, third-party trackers 172, location service 174, and client module(s) 176 for a remote monitoring service, such as HMS 22. Event engine 170 may be configured to predict a likelihood of a health event. Event engine 170 may be responsive to receipt of an alert transmission from IMD 10 indicating that IMD 10 detected a health event. As described herein, third-party trackers 172 record various attributes corresponding to communications with other people over connections with their devices. Location service 174 refers to a software module for receiving updates to a present location of computing device 12 in response to patient movement. Location service 174 may determine a current location of computing device 12 and that location can be presumed to be the latest known location of patient 4. Location service 174 may use global position system (GPS) data, multilateration, and/or any other known techniques for locating computing devices.

Client module(s) 176 may combine these updates with data captured by third-party trackers 172 and data recorded by client module(s) 176 and then, communicate in “heartbeats” datasets indicative of connectivity information 192 and location information 194 for HMS 22 to use in proactively alerting responders when the patient engages in risky behavior. Client module(s) 176 may control performance of any of the operations in response to detection of risky patient behavior ascribed herein to computing device 12, such as activating an alarm, rendering content 196 for display to the patient based on various data received from HMS 22, transmitting alert and heartbeat messages to HMS 22, controlling IoT devices 30, and analyzing data to confirm or override the detection of the health event by IMD 10.

Event engine 170 analyzes physiological data 190 for indicia of patient activities. Physiological data 190 includes sensed data from a number of sensors, patient input, and/or EHR data, to determine whether there is a sufficient likelihood that patient 4 is experiencing or is about to experience the health event detected by IMD 10. Physiological data 190 may include data received from IMD 10 as part of the alert transmission, additional data transmitted from IMD 10, e.g., in “real-time,” and physiological signals and other data related to the condition of patient 4 collected by computing device(s) 12 and/or IoT devices 30. In some examples, the sensed data from IMD 10 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient activities that occurred prior to the detection, e.g., as part long-term monitoring of the health of patient 4, may be recorded by client module(s) 176 as part of health monitoring application 150. Examples of recorded patient activity data may include one or more of: exercise and activity data, sleep data, symptom data, medical history data, quality of life data, nutrition data, medication taking or compliance data, allergy data, demographic data, weight, and height. EHR data may include any of the information regarding the historical condition or treatment(s) of patient 4 described above. EHR data may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data may also include demographic and other information of patient 4, such as age, gender, height, weight, and BMI.

FIG. 3B is a block diagram illustrating an example configuration of computing device 100 for an emergency medical response provider. Computing device 100 may be configured similarly to the configuration of computing device 12 illustrated in FIG. 3A except for application 151, which is capable of exchanging data from HMS 22 and different from health monitoring application.

In one example, application 101 may be a browser application for presenting, on a display to the responder, content 122 (e.g., Internet content) from HTML code downloaded from HMS 22. Content 122 may include map data depicting a geographic area with an indication for a latest patient location and an indication for a latest responder location. Via UI interface 160, application 151 may present an electronic form for the responder to enter various qualities to store as attribute data 124. Examples of such qualities include any achievement earned by the responder, such as qualifications, compliance status, and/or the like.

Application 151 may download and run monitoring service agent 142 in a background of userspace 102. Monitoring service agent 142 may automatically download content 122 when available from HMS 22 and upload attribute data 124 for HMS 22 to use in its ranking system for a plurality of responders. Monitoring service agent 142 may collect datasets of availability information 126 and location information 128 from various sources of patient activity indicia, including applications such as application 151, computing services such as a location service, responder input data submitted to HMS 22, and/or the like.

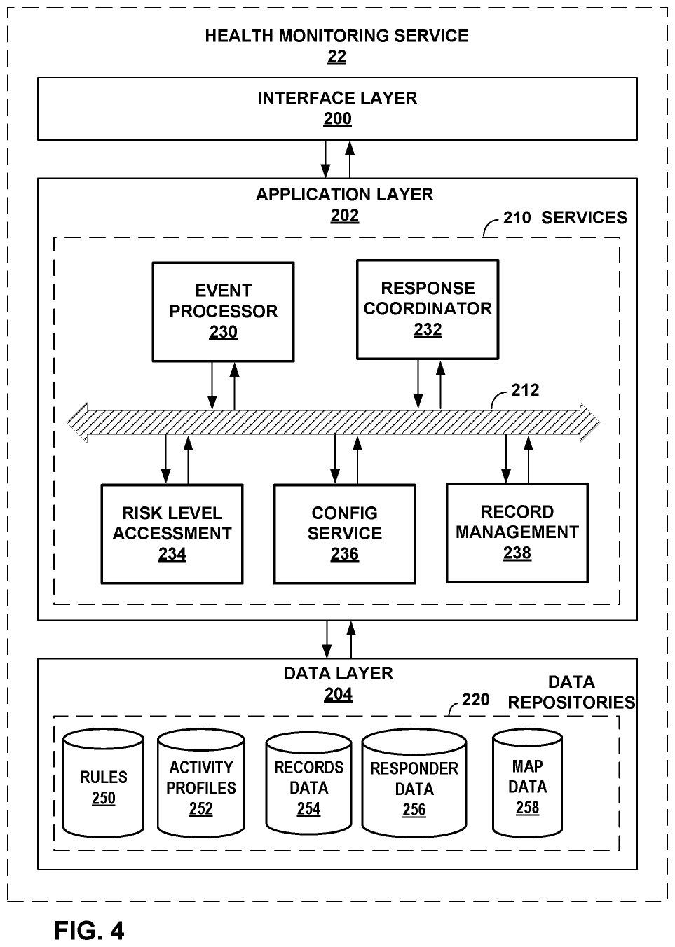

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, devices 48, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., an alert of an health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in a computing system using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of service module(s) 210 230-246 is implemented in a modular form within HMS 22. Although shown as separate modules for each service, in some examples the functionality of two or more services may be combined into a single module or component. Each of services 210 may be implemented in software, hardware, or a combination of hardware and software. Moreover, services 210 may be implemented as standalone devices, separate virtual machines or containers, processes, threads or software instructions generally for execution on one or more physical processors.

Event processor service 230 may be responsive to receipt of an alert transmission from computing device(s) 12 and/or IoT device(s) 30 indicating that IMD 10 detected a health event of patient and, in some examples, that the transmitting device confirmed the detection. Event processor service 230 may initiate performance of any of the operations in response to detection of a health event ascribed herein to HMS 22, such as communicating with patient 4, bystander 26, and responders 40, activating drone 46 and, in some cases, analyzing data to confirm or override the detection of the health event by IMD 10.

Record management service 238 may store the patient activity data or responder activity data included in a received heartbeat message within records data 254. Response coordinator service 232 may package the some or all of the data from records data 254, in some cases with additional information as described herein, into one more alert messages for transmission to bystander 26, responders 40, and/or devices 48 of connections. Responder data 256 may store data used by response coordinator service 232 to identify to whom to send alerts based on locations of potential responders 40 relative to a location of patient 4 and/or applicability of the care provided by responders 40 to the expected health event experienced by patient 4. Responder data 256 may include a ranking system for evaluating potential responders within a geographic area.

HMS 22 may advantageously utilize observed patient behaviors from one or more data sources, such as from connectivity and location information in data store 38 and/or patient information in IMD 10, for accurately estimating a level of risk that logically can be attributed to patient 4 of FIG. 1. Based on determining a reasonable prediction for the most likely patient activity at a current/present point-in-time and possibly, one or more other factors, HMS 22 may determine a current level of patient risk. HMS 22 may improve upon conventional patient risk assessment through a variety of methods/mechanisms. HMS 22 may measure “accuracy” (and therefore, improvements in accuracy) through different means. For example, HMS 22 may implement a model to predict the patient's risk level and then, perform example steps to improve upon an accuracy of that model, for instance, in terms of specificity and/or sensitivity.

For any given patient, risk level assessment service 234 may achieve a more accurate determination of a current risk level by performing one or more example steps as described herein. Applying different factors most likely will have at least a modicum of influence on the current level of patient risk. Adding more factors and/or selecting more informative features to use as factors of the current level of patient risk also have an effect on the logic used for the determination of the current patient risk level. Therefore, deciding which data point(s) to use for the patient risk assessment may substantially affect which level of risk is determined. It should be noted that risk level assessment service 234 may perform additional example steps towards achieving a more accurate patient risk assessment.

Risk level assessment service 234 may generate rules 250 to apply to a particular activity profile 252 for a patient being monitored and based on results from the application, determine a risk level of the patient for a given point-in-time. In some examples, rules 250 and the operation of risk level assessment service 234 as a rules engine may provide a more complex analysis of a patient activity profile 252 and various patient data. Risk level assessment service 234 may be configured to modify rules 250 based on feedback indicating whether the detections, predictions, and/or confirmations of risk levels (e.g., health events by IMD 10 and computing device 12) were accurate. The feedback may be received from patient 4, or from responders 40 and/or EHR 24 via HMS 22. In some examples, risk level assessment service 234 may utilize the datasets from true and false detections, predictions, and/or confirmations for supervised machine learning to further train models included as part of rules 250.

Rules 250 may include one or more models, algorithms, decision trees, and/or thresholds, which may be developed by risk level assessment service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In examples in which HMS 22 performs an analysis to confirm or override the prediction of the health event by IMD 10 and/or a determination of a current risk level for patient 4, event processor service 230 may apply one or more rules 250 to the data received in the alert message, e.g., to feature vectors derived by event processor service 230 from the data.

Risk level assessment service 234 may be configured to modify rules 250 based on event data 254 that indicates whether the detections and confirmations of health events by IMD 10, computing device 12, and/or HMS 22 were accurate. Similarly, rules configuration service 234 may be configured to modify rules 250 to determine highly accurate patient risk levels based on event data 254 that indicates whether the determinations and confirmations of the patient risk levels were correct.

Records data 254 may be received from patient 4, e.g., via computing device(s) 12, or from responders 40 and/or EHR 24. In some examples, rules configuration service 234 may utilize event records from true and false detections (as indicated by event data 254) and confirmations for supervised machine learning to further train models included as part of rules 250.

As illustrated in the example of FIG. 4, services 210 may also include a client application configuration service 236 for configuring and interacting with a software application implemented in computing device 12, computing device 100, or other computing devices. A medical application for patient health monitoring and heath event detection as described herein is one example software application; alternatively, a different software application may be configured by client application configuration service 236.

For example, configuration service 236 may provide the client applications context analyses to improve their operation over time. In some examples, configuration service 236 may apply machine learning techniques to analyze sensed data stored in event records 252, as well as the ultimate disposition of the event, e.g., indicated by EHR 24, to modify the operation of the client applications, e.g., for patient 4, a class of patients, all patients, or for particular users or devices, e.g., care givers, bystanders, etc.

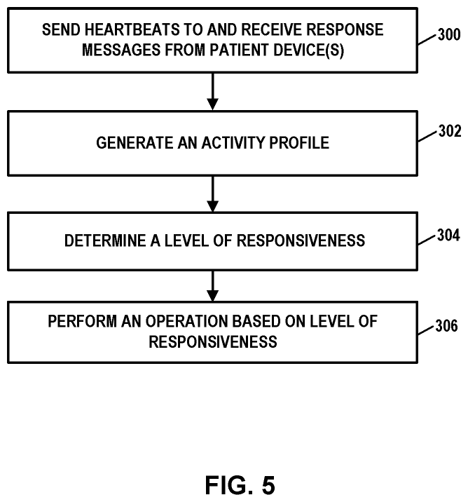

FIG. 5 is a flow diagram illustrating an example operation by a computing device of a health monitoring service that operates in accordance with one or more techniques of the present disclosure. The computing device may be one device of a number of different devices forming at least part of a computing system. The example operation depicted in FIG. 5 is described with respect to computing system 20A for running a health monitoring service known as HMS 22 as depicted in FIGS. 1 and 4, but may be described with respect to any computing service(s). The computing device of HMS 22 may send/receive various information corresponding to various human users (e.g., patients, responders, bystanders, and/or the like) via their personal device(s), e.g., computing devices 12, 42, or 48, other devices, e.g., IoT devices 30, network devices 34, AED 44, drone 46, and possibly, any number of additional devices.

The computing device of HMS 22 and computing device 12 depicted in FIG. 1 and FIG. 3A may share common hardware/software components, such as processing circuitry 130, memory 132, one or more input devices 134, one or more output devices 136, one or more sensors 146, and communication circuitry 140. The computing device of HMS 22 is described herein (in part) for using one or more of these hardware/software components to perform at least a portion of the example operation depicted in FIG. 5. The example operation depicted in FIG. 5 is described herein in terms of the one or more common hardware/software components.

According to the illustrated example of FIG. 5, processing circuitry of system 2 (e.g., processing circuitry of one or more computing device(s) in computing system 20A of FIG. 1) provides patient risk management resources based on a patient's behavior. The patient's activities over a time period are collected and recorded, for example, as connectivity and location information (e.g., the connectivity and location information in data store 38) generated by different devices from across one or more networks (e.g., network 16). Computing device 12 of FIG. 3A represents an example of such a device in which case, according to one example, computing device 12 sends to HMS 22 connectivity and location information 192 generated by processing circuitry 130 executing tracker(s) 172. In another example, computing device 12 provides HMS 22 with access to such information. Accordingly, upon determining availability of such information, the processing circuitry of system 2 is configured to subsequently retrieve and then, process connectivity and location information 192 generated by processing circuitry 130 executing tracker(s) 172.

The processing circuitry of system 2 may use alternative or additional data such as sensor data 82 generated by sensing circuitry 52 of IMD 10, patient data 190 generated by processing circuitry 130, tracking data generated by IoT devices 30, network history data generated by network devices 34, and/or the like. The processing circuitry of system 2 may, possibly, incorporate user input (e.g., patient input). According to the illustrated example of FIG. 5, the processing circuitry of system 2 determines a current risk level for a patient based on that patient's recent behavior. The processing circuitry of system 2 may use the above-mentioned connectivity and location information to identify/confirm one or more recent patient activities and then, access patient risk based on a number of factors.

A first computing device includes the processing circuitry of system 2 for the example operation of FIG. 5, which commences when the processing circuitry of system 2 periodically or asynchronously sends heartbeat messages to a second computing device of at least one of a patient or a responder (300). The set of heartbeat messages include requests for the patient or the responder to provide user input that causes the second computing device to send response messages that are respectively in response to the set of heartbeat messages.

After a sufficient number of heartbeat messages and/or response messages, the processing circuitry of system 2 generates, based at least in part on the set of heartbeat messages and the set of response messages, an activity profile of the at least one of the patient or the responder that indicates a level of responsiveness of the patient or the responder to respond to an acute health event of the patient (302). In one example, the processing circuitry of system 2 determines an amount of time that elapses between a particular heartbeat message and the response message. In such an example, the level of responsiveness is correlated to the amount of time that elapses between a particular heartbeat message and the response message. In another example, the processing circuitry of system 2 may be configured to identify, from the activity profile, responsive actions to the set of heartbeat messages and then, correlate the responsive actions with the set of response messages to determine a measure of responsiveness of the patient or the responder to the set of heartbeats

Based on the activity profile of the at least one of the patient or the responder, the processing circuitry of system 2 generates data that indicates a level of responsiveness of the patient or the responder to respond to an acute health event of the patient (304). In one example, the processing circuitry of system 2 may output, to the second computing device of the at least one of the patient or the responder or a third device, content data for rendering a map that displays the level of responsiveness of one or more responders proximate to the patient.

To improve an effectiveness of the response to the acute health event, the processing circuitry of system 2 performs at least one operation based at least in part on the level of responsiveness of the patient or the responder to respond to the acute health event (306). These operations include sending proactive alerts and coordinating responses to acute health events of patient 4. For example, in response to the first computing device detecting the acute health event, the processing circuitry of system 2 is configured to select one or more responders, each of which with a level of responsiveness that is greater than a threshold, and then, send a notification to each of the one or more responders to provide care to the patient experiencing the acute health event. In response to determining from the activity profile that the level of responsiveness of the patient or the responder to respond to the acute health event does not satisfy a threshold, the processing circuitry of system 2 is configured to send a notification to the patient or responder to change their behavior in a way that increases their corresponding level of responsiveness. In response to determining from the activity profile that the level of responsiveness of the patient or the responder to respond to the acute health event does not satisfy a threshold, the processing circuitry of system 2 is configured to send a notification to each person with whom the patient has a social relationship of a lack of responsiveness from the patient.

To commence proactive patient risk management of the example operation of FIG. 5, the processing circuitry of system 2 may correlate connectivity and location information from a plurality of sources across multiple networks including other computing services, and then, generate an activity profile for a patient based on the correlated information. The variety of devices that the processing circuitry may employ as sources include a mobile device (e.g., a smartphone or smartwatch), a desktop computer, or a smart television.

At a later point-in-time, the processing circuitry determines a current risk level to attribute to recent patient behavior as indicated by the activity profile and various patient data. System 2 may employ a number of technologies (e.g., trackers running in the patient's mobile phone, a smart speaker device, and/or IMD 12 itself) to collect information regarding patient's activities including any connectivity and location information as described herein. The processing circuitry may engage with the patient and illicit patient input, for example, to assess the patient's risk level or refine a previous assessment of the current risk level.

In response to the determination of the current risk level, the processing circuitry communicates, via communication circuitry, the activity profile and the current risk level to the patient via their personal device (i.e., patient device) (304). The processing circuitry may communicate this information in a notification message directed to the patient device with the intent of bringing the activity profile and the current risk level to the attention of the patient. According to a schedule, the processing circuitry may be programmed to (e.g., periodically) distribute the above information to one or more of the patient's devices, for example, in the form content to be displayed to the patient on a display device. UI component of computing device 12 may render the content and present the information on the display device.

In an alternative example, the processing circuitry may distribute the content in response to a request or a query from patient or a caregiver. For instance, the patient may submit the request to HMS 22 via a client application, triggering the generation and subsequent distribution of the above content.

Based on the current risk level, the processing circuitry may communicate an alert to one or more potential responders (306). These responders may include any available emergency medical personnel within a pre-determine proximity to the at-risk patient. Because the current risk level is an accurate indicator of the patient's overall health, the processing circuitry may use the current risk level to predict likely changes in patient health. If the processing circuitry determines that an acute health event is likely to occur in a near future and/or expected to cause harmful physical damage to the patient, the processing circuitry may notify the patient and the patient's caregiver regarding the acute health event. The processing circuitry may identify a medical professional with requisite qualifications for preventing the acute health event in the patient and/or remedying the physical damage. Therefore, the processing circuitry may use the current risk level to benefit the patient by detecting possible health events to occur in the future and coordinating a response to avoid or mitigate these health events.

To illustrate by way of example, the patient may be at a high-risk level if the patient is experiencing, has recently experienced, or is projected to experience a health event. Similar to the activity profile, the processing circuitry may perform a determination as to whether patient physiological data (e.g., sensor signals from IMD 10) is indicative of the health event. The processing circuitry may determine that physiological data of the patient is indicative of a health event, such as a sudden cardiac arrest, and responsive to that determination, coordinate an immediate response to bring medical attention to the patient. In some examples, the processing circuitry makes this determination based on receiving an alert message from another device. In some examples, the processing circuitry analyzes sensed physiological data to determine whether sudden cardiac arrest is detected. As described herein, the processing circuitry may employ a rules engine (e.g., rules engine 74 and/or rules engine 172) to apply various rules (e.g., rules 84 or rules 196) to the sensed physiological data in order to determine whether an health event has occurred, is occurring, or is about to occur. In general, each rule sets forth one or more conditions (e.g., minimum or maximum thresholds, ranges, qualities, quantities, and/or the like for parameter values) that, if true, qualify the sensed physiological data as sufficient evidence of an imminent or an occurring health event, such as a sudden cardiac arrest.

In some examples, IMD 10 and/or computing device(s) 12 may generate an alert in the form of an auditory alarm and/or or a message communicated to a healthcare provider. In some examples, IMD 10 and/or computer device(s) 12 may trigger the alert by communicating a control directive for a third device to activate an alarm or another alert type. In some examples, the processing circuitry may request a response from medical professionals including a doctor, a clinician, and/or emergency medical services (EMS).

In some instances, the processing circuitry may modify a previous risk level assessment and determination of the current risk level. The output device, in turn, may generate output data indicative of a modified determination based on various criteria (e.g., cancellation criteria). The modified determination may indicate a false rejection, a false detection, a true rejection, or a true rejection of the sudden cardiac arrest. In some examples, the processing circuitry computes or modifies a likelihood of the sudden cardiac arrest based on the sensed physiological data and other signals.

To further illustrate, via the output device and/or an output device of another computing device, the processing circuitry may cause an ongoing alert (e.g., alarm) indicating that an emergency has occurred, and emergency medical services (EMS) have been summoned. The processing circuitry may trigger the ongoing alert or activate the same in the patient device or the patient's medical device.

The processing circuitry of system 2 may communicate a message, via a network connection, to the EMS informing that healthcare provider of the patient's health event and/or high-risk level. In other examples, the processing circuitry may cancel the ongoing alert of the sudden cardiac arrest. In addition to the cancellation of the alarm, the processing circuitry 12 may generate and then, communicate to, one or more healthcare providers, messages conveying that the patient is at-risk (e.g., high risk) of experiencing a sudden cardiac arrest or other health event. An example message may indicate that the alarm was a false alarm, or that the sudden cardiac arrest determination is a true detection but that the patient has recovered or is stable. An example message may prompt a user or device to alter the patient's therapy (e.g., to start/stop CPR). Another example message may prompt a user or device to remove a specific treatment. Another example message may be communicated to the EMS in order to redirect an ambulance to a specialized care center or another caregiver. Another example message may be communicated to a user or device to trigger a specific treatment (for example, instruct the patient or bystander to utilize an AED, find the location of a nearby drone-delivered AED, take medication or another treatment, and/or the like) and then, add that specific treatment to the patient's therapy.

The processing circuitry of system 2 may perform a number of additional/alternative steps of the example operation illustrated in FIG. 5. For example, a current location for the patient may be abnormally out of range/out of network, such as in another country (e.g., out of country) where the patient's presence constitutes risky behavior. The patient may be considered “out-of-country” notwithstanding any active connection or connection loss with HMS 22. If the processing circuitry of system 2 determines that the patient has traveled to a foreign country, the processing circuitry may allow HMS 22 to coordinate safety protocols for the patient. This may include a variety of services, including automatically contacting the patient's insurance company to notify them of the patient's travel to the other county. The insurance company may proceed to communicate information to the patient and any caregiver of the patient.

The processing circuitry of system 2 may enable HMS 22 to trigger the above protocol if the patient is determined to be outside a “normal” coverage area for HMS 22. The processing circuitry of system 2 may allow the patient to switch to different entities for remote health monitoring and health event detection/prevention. Another protocol may use a colored indicator to notify the patient of a shut down to the patient's device (e.g., where, at best, only authorized calls permitted). Another protocol may direct HMS 22 to generate, for display to the patient via the patient device, a map depicting locations/objects/people of interest such as a pulsepoint person and/or an AED within a pre-determined proximity.

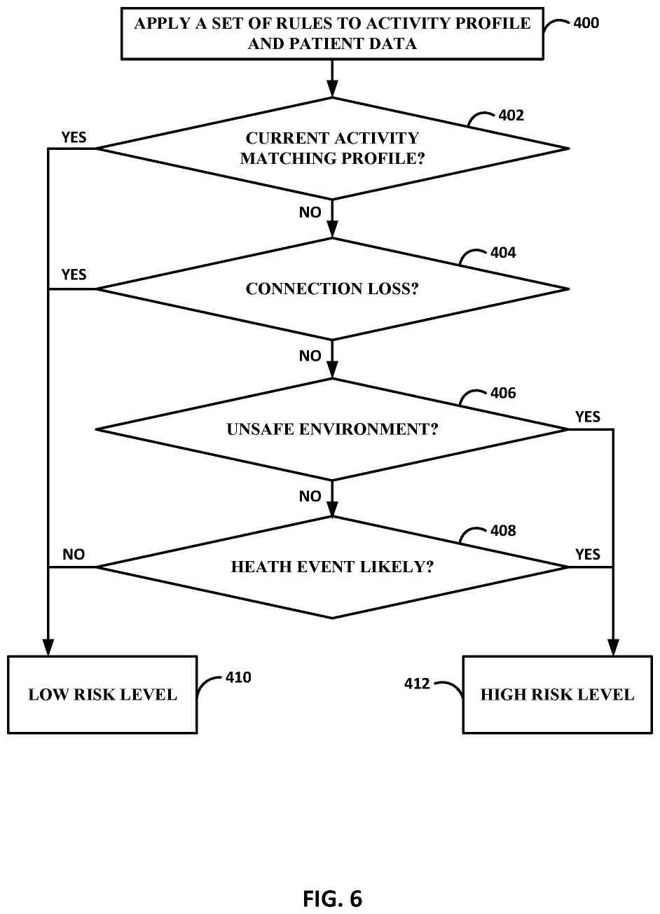

FIG. 6 is a flow diagram illustrating an example operation by computing device(s) 12 (that operates in accordance with one or more techniques of the present disclosure. Although described with respect to computing device(s) 12, the example of FIG. 6 may be performed by any one or more of computing devices 12, 42, or 48, IoT devices 30, AED 44, or drone 46. The example operation depicted in FIG. 6 may be described with respect to system 2 of FIGS. 1-4.

According to the illustrated example of FIG. 6, processing circuitry of system 2 applies a set of rules to an activity profile and various patient data (400). Subsequent steps of the example operation proceed to apply the set of rules.

According to a first rule, the processing circuitry of system 2 performs a determination as to whether current patient activity matches a corresponding activity of the activity profile (402). The corresponding activity may be identified in the profile as the most likely patient activity at a current time based on past behavior. Based on a determination that the current patient activity matches the activity profile (YES of 402), the processing circuitry of system 2 finalizes the rule application and sets a low risk level as the current risk level for the patient (410). Based on a determination that the current patient activity fails to match the activity profile (NO of 402), the processing circuitry of system 2 proceeds to apply a next rule of the set of rules.

According to a second rule, the processing circuitry of system 2 performs a determination as to whether a connection has been lost with a patient device (404). Based on a determination that the connection has been lost (YES of 404), the processing circuitry of system 2 finalizes the rule application and establishes a low risk level as the current risk level for the patient (410). Based on a determination that the connection has not been lost (i.e., no connection loss detected) (YES of 404), the processing circuitry of system 2 proceeds to apply a next rule of the set of rules.

According to a third rule, the processing circuitry of system 2 performs a determination as to whether the patient is currently located in or travelling to an unsafe environment (e.g., geographic area of any size). Based on a determination that a current patient location or a projected/future patient location corresponds to an unsafe environment for the patient (YES of 406), the processing circuitry of system 2 finalizes the rule application and establish a high risk level as the current risk level for the patient (412). Based on a determination that a current patient location or a projected/future patient location corresponds to a safe environment for the patient (NO of 406), the processing circuitry of system 2 proceeds to apply a next rule of the set of rules.

According to a fourth rule, the processing circuitry of system 2 performs a determination as to whether a health event is likely to occur, is occurring, or has recently occurred. Based on a determination that the health event is not likely and/or has not occurred (NO of 408), the processing circuitry of system 2 finalizes the rule application and establishes a low risk level as the current risk level for the patient (410). Based on a determination that the health event is likely to occur and/or has recently occurred (YES of 408), the processing circuitry of system 2 finalizes the rule application and establish a high-risk level as the current risk level for the patient (412).

Example 1. A first computing device comprising: processing circuitry; and a memory comprising instructions that, when executed by the processing circuitry, cause the processing circuitry to: at least one of periodically or asynchronously send heartbeat messages to a second computing device of at least one of a patient or a responder, the set of heartbeat messages comprising requests for the patient or the responder to provide user input that causes the second computing device to send response messages that are respectively in response to the set of heartbeat messages; generate, based at least in part on the set of heartbeat messages and the set of response messages, an activity profile of the at least one of the patient or the responder that indicates a level of responsiveness of the patient or the responder to respond to an acute health event of the patient; and perform at least one operation based at least in part on the level of responsiveness of the patient or the responder to respond to the acute health event.

Example 2. The first computing device of Example 1, wherein the memory further comprises instructions that cause the processing circuitry to: determine an amount of time that elapses between a particular heartbeat message and the response message, wherein the level of responsiveness is correlated to the amount of time that elapses between a particular heartbeat message and the response message.

Example 3. The first computing device of Example 1 or Example 2, wherein the memory further comprises instructions that cause the processing circuitry to: output, to a third device or the second computing device of the at least one of the patient or the responder, content data for rendering a map that displays the level of responsiveness of one or more responders proximate to the patient.

Example 4. The first computing device of any of Examples 1-3, wherein the memory further comprises instructions that cause the processing circuitry to: in response to the first computing device detecting the acute health event, select one or more responders, each of which with a level of responsiveness that is greater than a threshold, and send a notification to each of the one or more responders to provide care to the patient experiencing the acute health event.

Example 5. The first computing device of any of Examples 1-4, wherein the memory further comprises instructions that cause the processing circuitry to: select the one or more responders based on a distance between each responder and the patient or available and connectivity information of the second computing device.

Example 6. The first computing device of any of Examples 1-5, wherein the memory further comprises instructions that cause the processing circuitry to: in response to determining from the activity profile that the level of responsiveness of the patient or the responder to respond to the acute health event does not satisfy a threshold, send a notification to the patient or responder to change their behavior in a way that increases their corresponding level of responsiveness.

Example 7. The first computing device of any of Examples 1-6, wherein to perform the at least one operation, the memory further comprises instructions that cause the processing circuitry to: in response to determining from the activity profile that the level of responsiveness of the patient or the responder to respond to the acute health event does not satisfy a threshold, send a notification to each person with whom the patient has a social relationship of a lack of responsiveness from the patient.

Example 8. The first computing device of any of Examples 1-7, wherein to generate the activity profile to predict a likely activity of the patient at time data, the memory further comprises instructions that cause the processing circuitry to: identify, from the activity profile, responsive actions to the set of heartbeats; and correlate the responsive actions with the set of response messages to determine a measure of responsiveness of the patient to the set of heartbeats.

Example 9. The first computing device of any of Examples 1-8, wherein one or more of the set of response messages comprises at least one of connectivity and location information of the patient or availability and location information of the responder.

Example 10. A computing device comprising: an input device; an output device; processing circuitry; and a memory comprising instructions that, when executed by the processing circuitry, cause the processing circuitry to: correlate connectivity and location information received via the input device from computing services running in a plurality of devices across at least one computer network, wherein at least one of the devices comprises a device of a patient that is configured to send an alert of a health event of the patient to at least one other device of the plurality of devices; generate an activity profile of the patient based on the correlated information, wherein the activity profile comprises likely activities of the patient based on time data; based on the activity profile of the patient, determine a level of responsiveness in event of the alert of the health; and communicate, via the output device, information of the level of responsiveness to the patient via the device of the patient.

Example 11. The computing device of Example 10, wherein at least one of a medical device or the second computing device of the patient is configured to send an alert of the acute health event of the patient to the first computing device, wherein the memory further comprises instructions that cause the processing circuitry to: responsive to the alert of the health event, identify one or more responders for the patient based a proximity of each responder to a location of the patient; and communicate, to each of the one or more responders, the information of the level of responsiveness to the patient via a device of that responder.

Example 12. The computing device of Example 11, wherein the information comprises a map of a geographic area having at least one indication of at least one patient location, the map comprising at least one of a spatial graph of recorded patient activity, a temporal graph for the recorded patient activity over a time period, or a social graph of people in the geographic area with whom the patient has a social relationship.

Example 13. The computing device of any of Examples 9-12, wherein the information comprises information of high-risk behavior, a notification of an unsafe environment, a notification of unusual patient activity, a notification of an impending health event, or a notification of a connection loss.

Example 14. The computing device of any of Examples 9-13, wherein the memory further comprises instructions that cause the processing circuitry to: determine the level of responsiveness based on indicia of responsivity in patient data, wherein the indicia of responsivity is determined from a comparison, for one or more likely activities of the patient determined from the activity profile, between each likely activity and an expected activity at a same time data.

Example 15. The computing device of any of Examples 9-14, wherein the memory further comprises instructions that cause the processing circuitry to: generate a set of rules configured to determine the level of responsiveness of the patient based on time data and the activity profile, wherein the activity profile is configured to identify a likely activity of the patient for the time data; determine the level of responsiveness of the patient based on an application of the set of rules to patient data; and communicate content for display on a display device to the patient via the device of the patient or to at least one second person via each respective device of the at least one second person.

Example 16. The computing device of any of Examples 9-15, wherein the memory further comprises instructions that cause the processing circuitry to: communicate, via the output device, the information of the level of responsiveness to a person different from the patient in response to determining that the level of responsiveness fails to satisfy a threshold; identify a responder for the patient based on the determining that the level of responsiveness fails to satisfy the threshold; and responsive to the alert of the health event from the device of the patient, communicate a notification to at least one of the patient, one or more responders including the identified responder, or the person different from the patient.

Example 17. The computing device of Example 16, wherein the person different from the patient is a caregiver of the patient, a person located within a pre-determined proximity of the patient, or a person with a social relationship to the patient, or a bystander.

Example 18. The computing device of Example 16, wherein the responder is a medical professional having a level of responsiveness that satisfies a criterion associated with patients failing to satisfy the threshold.

Example 19. The computing device of Example 16, wherein the memory further comprises instructions that cause the processing circuitry to: determine that the patient and the device of the patient are in different locations; and communicate a notification to the responder that the patient is not in possession of the device of the patient.

Example 20. The computing device of any of Examples 10-19, wherein memory further comprises instructions that, when executed by the processing circuitry, cause the processing circuitry to: send, over a network, a message for verifying a connection with the patient via the device of the responder; and receive, over the network, a reply message in return to the message, wherein the message response is a confirmation of the connection between the computing device and the device of the patient.

Example 21. The computing device of Example 20, wherein memory further comprises instructions that, when executed by the processing circuitry, cause the processing circuitry to: for a plurality of messages, repeat an operation to send the message and an operation to receive the reply message.

Example 22. A computing device comprising: an input device; an output device; processing circuitry; and a memory comprising instructions that, when executed by the processing circuitry, cause the processing circuitry to: correlate availability and location information received via the input device from computing services running in a plurality of devices across at least one computer network, wherein at least one of the devices comprises a device of a patient that is configured to send an alert of a health event of the patient, and wherein at least one of the devices comprises a device of a responder that is configured to receive the alert of the health event of the patient; generate an activity profile of the responder based on the correlated information, wherein the activity profile comprises likely activities of the responder based on time data; based on the activity profile of the responder, determine a level of responsiveness in event of the alert of the health; and communicate, via the output device, information of the level of responsiveness to the responder via the device of the responder.

Example 23. The computing device of Example 22, wherein the memory further comprises instructions that, when executed by the processing circuitry, cause the processing circuitry to: identify the responder for the patient based on one or more qualitative measures for respective ones of a plurality of candidate responders.

Example 24. The computing device of Example 22 or 23, wherein memory further comprises instructions that, when executed by the processing circuitry, cause the processing circuitry to: communicate, via the output device, content for display to the identified responder via the device of the responder, the content comprises a notification that a level of responsiveness of the patient falls below a minimum threshold.

Example 25. The computing device of any of Examples 22-24, wherein the content comprises a map of a geographic area having at least one indication of at least one patient location.

Example 26. The computing device of any of Examples 22-25, wherein memory further comprises instructions that, when executed by the processing circuitry, cause the processing circuitry to: for a sequence of messages, send, over a network, a message for verifying a connection with the responder via the device of the responder, and receive, over the network, a response message in reply to the message, wherein the message comprises a confirmation of the connection between the computing device and the device of the responder.

Example 27. The computing device of Example 26 wherein each response message indicates a current availability to serve as an emergency responder for the patient.

Example 28. A method for operating processing circuitry, the method comprising, by processing circuitry: at least one of periodically or asynchronously sending heartbeat messages to a computing device of at least one of a patient or a responder, the set of heartbeat messages comprising requests for the patient or the responder to provide user input that causes the computing device to send response messages that are respectively in response to the set of heartbeat messages; generate, based at least in part on the set of heartbeat messages and the set of response messages, an activity profile of the at least one of the patient or the responder that indicates a level of responsiveness of the patient or the responder to respond to an acute health event of the patient; and perform at least one operation based at least in part on the level of responsiveness of the patient or the responder to respond to the acute health event.

Example 29. A method for operating processing circuitry of a computing system coupled to a device of a human user comprising, by processing circuitry: send, over a network, a heartbeat to the a computing device of the a human user, wherein the heartbeat message corresponds to a connection check with the computing device device of the human user; and receive, over the network, a reply to the heartbeat, wherein the reply comprises an indication of a stable connection between the computing system and the computing device of the human user.

Example 30. A system comprising processing circuitry configured to: at least one of periodically or asynchronously send heartbeat messages to a computing device of at least one of a patient or a responder, the set of heartbeat messages comprising requests for the patient or the responder to provide user input that causes the computing device to send response messages that are respectively in response to the set of heartbeat messages; generate, based at least in part on the set of heartbeat messages and the set of response messages, an activity profile of the at least one of the patient or the responder that indicates a level of responsiveness of the patient or the responder to respond to an acute health event of the patient; and perform at least one operation based at least in part on the level of responsiveness of the patient or the responder to respond to the acute health event.

Example 31. The system of Example 30, wherein the computing device comprises at least one of a smartphone, a smartwatch, a smart speaker, a smart television, an Internet of Things (IoT) device, or a network access point.

Example 32. A non-transitory computer readable storage medium comprising program instructions configured to cause processing circuitry to: at least one of periodically or asynchronously send heartbeat messages to a computing device of at least one of a patient or a responder, the set of heartbeat messages comprising requests for the patient or the responder to provide user input that causes the computing device to send response messages that are respectively in response to the set of heartbeat messages; generate, based at least in part on the set of heartbeat messages and the set of response messages, an activity profile of the at least one of the patient or the responder that indicates a level of responsiveness of the patient or the responder to respond to an acute health event of the patient; and perform at least one operation based at least in part on the level of responsiveness of the patient or the responder to respond to the acute health event.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims.

1. A first computing device comprising:
processing circuitry; and
a memory comprising instructions that, when executed by the processing circuitry, cause the processing circuitry to:
at least one of periodically or asynchronously send heartbeat messages to a second computing device of at least one of a patient or a responder, the set of heartbeat messages comprising requests for the patient or the responder to provide user input that causes the second computing device to send response messages that are respectively in response to the set of heartbeat messages;
generate, based at least in part on the set of heartbeat messages and the set of response messages, an activity profile of the at least one of the patient or the responder that indicates a level of responsiveness of the patient or the responder to respond to an acute health event of the patient; and
perform at least one operation based at least in part on the level of responsiveness of the patient or the responder to respond to the acute health event.

2. The first computing device of claim 1, wherein the memory further comprises instructions that cause the processing circuitry to:
determine an amount of time that elapses between a particular heartbeat message and the response message, wherein the level of responsiveness is correlated to the amount of time that elapses between a particular heartbeat message and the response message.

3. The first computing device of claim 1, wherein the memory further comprises instructions that cause the processing circuitry to:
output, to a third device or the second computing device of the at least one of the patient or the responder, content data for rendering a map that displays the level of responsiveness of one or more responders proximate to the patient.

4. The first computing device of claim 1, wherein the memory further comprises instructions that cause the processing circuitry to:
in response to the first computing device detecting the acute health event, select one or more responders, each of which with a level of responsiveness that is greater than a threshold, and send a notification to each of the one or more responders to provide care to the patient experiencing the acute health event.

5. The first computing device of claim 1, wherein the memory further comprises instructions that cause the processing circuitry to:
select the one or more responders based on a distance between each responder and the patient or available and connectivity information of the second computing device.

6. The first computing device of claim 1, wherein the memory further comprises instructions that cause the processing circuitry to:
in response to determining from the activity profile that the level of responsiveness of the patient or the responder to respond to the acute health event does not satisfy a threshold, send a notification to the patient or responder to change their behavior in a way that increases their corresponding level of responsiveness.

7. The first computing device of claim 1, wherein to perform the at least one operation, the memory further comprises instructions that cause the processing circuitry to:
in response to determining from the activity profile that the level of responsiveness of the patient or the responder to respond to the acute health event does not satisfy a threshold, send a notification to each person with whom the patient has a social relationship of a lack of responsiveness from the patient.

8. The first computing device of claim 1, wherein to generate the activity profile to predict a likely activity of the patient at time data, the memory further comprises instructions that cause the processing circuitry to:
identify, from the activity profile, responsive actions to the set of heartbeats; and
correlate the responsive actions with the set of response messages to determine a measure of responsiveness of the patient to the set of heartbeats.

9. The first computing device of claim 1, wherein one or more of the set of response messages comprises at least one of connectivity and location information of the patient or availability and location information of the responder.

10. A computing device comprising:
an input device;
an output device;
processing circuitry; and
a memory comprising instructions that, when executed by the processing circuitry, cause the processing circuitry to:
correlate connectivity and location information received via the input device from computing services running in a plurality of devices across at least one computer network, wherein at least one of the devices comprises a device of a patient that is configured to send an alert of a health event of the patient to at least one other device of the plurality of devices;
generate an activity profile of the patient based on the correlated information, wherein the activity profile comprises likely activities of the patient based on time data;
based on the activity profile of the patient, determine a level of responsiveness in event of the alert of the health; and
communicate, via the output device, information of the level of responsiveness to the patient via the device of the patient.

11. A computing device comprising:
an input device;
an output device;
processing circuitry; and
a memory comprising instructions that, when executed by the processing circuitry, cause the processing circuitry to:
correlate availability and location information received via the input device from computing services running in a plurality of devices across at least one computer network, wherein at least one of the devices comprises a device of a patient that is configured to send an alert of a health event of the patient, and wherein at least one of the devices comprises a device of a responder that is configured to receive the alert of the health event of the patient;
generate an activity profile of the responder based on the correlated information, wherein the activity profile comprises likely activities of the responder based on time data;
based on the activity profile of the responder, determine a level of responsiveness in event of the alert of the health; and
communicate, via the output device, information of the level of responsiveness to the responder via the device of the responder.

12. A method for operating processing circuitry comprising:
at least one of periodically or asynchronously sending heartbeat messages to a computing device of at least one of a patient or a responder, the set of heartbeat messages comprising requests for the patient or the responder to provide user input that causes the computing device to send response messages that are respectively in response to the set of heartbeat messages;
generate, based at least in part on the set of heartbeat messages and the set of response messages, an activity profile of the at least one of the patient or the responder that indicates a level of responsiveness of the patient or the responder to respond to an acute health event of the patient; and
perform at least one operation based at least in part on the level of responsiveness of the patient or the responder to respond to the acute health event.

13. A method for operating processing circuitry of a computing system:
send, over a network, a heartbeat to a computing device of a human user, wherein the heartbeat message corresponds to a connection check with the computing device of the human user; and
receive, over the network, a reply to the heartbeat, wherein the reply comprises an indication of a stable connection between the computing system and the computing device of the human user.

14. A system comprising processing circuitry configured to:
at least one of periodically or asynchronously send heartbeat messages to a computing device of at least one of a patient or a responder, the set of heartbeat messages comprising requests for the patient or the responder to provide user input that causes the computing device to send response messages that are respectively in response to the set of heartbeat messages;
generate, based at least in part on the set of heartbeat messages and the set of response messages, an activity profile of the at least one of the patient or the responder that indicates a level of responsiveness of the patient or the responder to respond to an acute health event of the patient; and
perform at least one operation based at least in part on the level of responsiveness of the patient or the responder to respond to the acute health event.

15. A non-transitory computer readable storage medium comprising program instructions configured to cause processing circuitry to:
at least one of periodically or asynchronously send heartbeat messages to a computing device of at least one of a patient or a responder, the set of heartbeat messages comprising requests for the patient or the responder to provide user input that causes the computing device to send response messages that are respectively in response to the set of heartbeat messages;
generate, based at least in part on the set of heartbeat messages and the set of response messages, an activity profile of the at least one of the patient or the responder that indicates a level of responsiveness of the patient or the responder to respond to an acute health event of the patient; and
perform at least one operation based at least in part on the level of responsiveness of the patient or the responder to respond to the acute health event.

16. The computing device of claim 10, wherein at least one of a medical device or the second computing device of the patient is configured to send an alert of the acute health event of the patient to the first computing device,
wherein the memory further comprises instructions that cause the processing circuitry to:
responsive to the alert of the health event, identify one or more responders for the patient based a proximity of each responder to a location of the patient; and
communicate, to each of the one or more responders, the information of the level of responsiveness to the patient via a device of that responder.

17. The computing device of claim 10, wherein the memory further comprises instructions that cause the processing circuitry to:
generate a set of rules configured to determine the level of responsiveness of the patient based on time data and the activity profile, wherein the activity profile is configured to identify a likely activity of the patient for the time data;
determine the level of responsiveness of the patient based on an application of the set of rules to patient data; and
communicate content for display on a display device to the patient via the device of the patient or to at least one second person via each respective device of the at least one second person.

18. The computing device of claim 10, wherein the memory further comprises instructions that cause the processing circuitry to:
communicate, via the output device, the information of the level of responsiveness to a person different from the patient in response to determining that the level of responsiveness fails to satisfy a threshold;
identify a responder for the patient based on the determining that the level of responsiveness fails to satisfy the threshold; and
responsive to the alert of the health event from the device of the patient, communicate a notification to at least one of the patient, one or more responders including the identified responder, or the person different from the patient.

19. The computing device of claim 18, wherein the memory further comprises instructions that cause the processing circuitry to:
determine that the patient and the device of the patient are in different locations; and
communicate a notification to the responder that the patient is not in possession of the device of the patient.

20. The computing device of claim 11, wherein memory further comprises instructions that, when executed by the processing circuitry, cause the processing circuitry to:
for a sequence of messages,
send, over a network, a message for verifying a connection with the responder via the device of the responder, and
receive, over the network, a response message in reply to the message, wherein the message comprises a confirmation of the connection between the computing device and the device of the responder.
